(19) United States
(12) Patent Application Publication
(10) Pub. No.: US 2006/0210999 A1
(43) Pub. Date: Sep. 21, 2006

(54) Microarray and method for genotyping SHV beta lactamases

(76) Inventors: Verena Grimm, Fellbach (DE); Milorad Susa, Stuttgart (DE); Cornelius Knabbe, Stuttgart (DE); Rolf D. Schmid, Stuttgart (DE); Till T. Bachmann, Stuttgart (DE)

Correspondence Address: Bell, Boyd & Lloyd LLC, P.O. Box 1135, Chicago, IL, 60690-1135, US

(21) Appl. No.: 11/083,788

(22) Filed: Mar. 18, 2005

(51) Int. Cl. C12Q 1/68

(52) U.S. Cl. 435/6

(57) ABSTRACT

The present invention relates to a method for genotyping SHV beta lactamase and a method for determining antibiotic resistance conferred by a SHV-type Extended Spectrum Beta Lactamase of a bacterium. Moreover, the present invention pertains to an oligonucleotide array and a kit for a genotyping based detection of SHV beta lactamases, as well as to a method for designing improved oligonucleotide capture probes.

### FIELD OF THE INVENTION

The present invention relates to a method for genotyping SHV beta lactamase and to a method for determining antibiotic resistance conferred by a SHV-type Extended Spectrum Beta Lactamase. Moreover, the present invention pertains to an oligonucleotide array and a kit for a genotyping based detection of SHV beta lactamases, as well as to a method for designing improved oligonucleotide capture probes.

### BACKGROUND OF THE INVENTION

Microbial resistance against antibiotics is an increasing threat to the favorable outcome of the antibiotic treatment of clinical or community acquired infections. In particular, numerous outbreaks of infections with organisms producing extended spectrum beta lactamases (ESBL) have been observed worldwide. The Extended Spectrum Beta-Lactamases (ESBLs) are plasmid mediated serine beta-lactamases and belong to the most relevant antibiotic resistance determinants in gram negative bacteria (Bradford, P. A. 2001. Extended-spectrum beta-lactamases in the 21st century: characterization, epidemiology, and detection of this important resistance threat. Clin. Microbiol. Rev. 14:933-51, Table). Most of them are derivatives of the classical TEM or SHV enzymes differing from the parental sequences (SHV-1 or TEM-1) by a few amino acid substitutions, which cause an increased spectrum of activity, especially e.g. against oxyimino-cephalosporins. Other derivatives of the classical TEM or SHV enzymes are resistant to inhibition to clavulanic acid and sulbactam and thus are called inhibitor resistant (IRT).

Clinical standard methods for a resistance detection as currently generally performed rely on phenotypic screening tests based on the inhibition of bacterial growth in disc diffusion tests or dilution tests. As is well known in the art, these standard tests are associated with several serious drawbacks. They require about 2 days before a positive or a negative test result may be obtained on the basis of which a specific antibiotic therapy can be provided to a patient or before a patient may be identified as a carrier of bacteria resistant to common antibiotics. Moreover, in case the isolates are also screened with respect to multiple antibiotics in combination with beta-lactamase inhibitors, the test results will be obtained generally only after 3 days, so that a treatment of a patient often has to start before the phenotype of the pathogenic bacterium is unequivocally determined.

In particular, the phenotypic detection of ESBL-type beta lactamases is complicated, time consuming and bears the risk of misinterpretation possibly resulting in a failure of treatment and spread of resistance. Especially due to the differing substrate spectra and the inoculum effect, an ESBL phenotype may remain undetected. Therefore, a confirmatory test has to be performed in addition to the routinely performed ESBL screening tests in order to allow an accurate ESBL detection. However the problem of limited sensitivity and the requirement of an organism isolation followed by overnight cultivation remains for all phenotypic confirmatory tests.

Moreover, the above tests of the state of the art are rather labor intensive and require highly skilled personal. Difficulties associated with the subjective interpretation of the test results may give raise to an incorrect diagnosis and thus to an ineffective treatment of a patient or to a spreading of bacteria resistant to common antibiotics.

### SUMMARY OF THE INVENTION

The above-mentioned problems are solved by the present invention by the provision of a method for genotyping SHV beta lactamase by determining the presence or absence of at least one single nucleotide polymorphism, said method comprising the steps of: (a) providing target DNA of SHV beta lactamase to be analyzed; (b) amplifying and labeling said target DNA; (c) fragmenting said amplified and labeled target DNA into smaller fragments; (d) contacting said fragments with an array of oligonucleotide capture probes, said array of oligonucleotide capture probes having at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe; (e) hybridizing said fragments of target DNA to said array of oligonucleotide probes; (f) determining the presence of a hybridization of a target DNA fragment to said at least one first oligonucleotide probe, said hybridization to said first oligonucleotide capture probe being indicative for the presence of SHV beta lactamase without single nucleotide polymorphism at the position of said at least one nucleotide of interest; (g) determining the presence of a hybridization of a target DNA fragment to said at least one second oligonucleotide probe, said hybridization to said second oligonucleotide capture probe being indicative for the presence of a SHV beta lactamase having said at least one single nucleotide polymorphism at the position of said at least one nucleotide of interest; and (h) determining the presence or absence of a specific genotype of SHV beta lactamase on the basis of the results of steps (f) and (g).

Moreover, there is provided a method of determining antibiotic resistance conferred by a SHV type Extended Spectrum Beta Lactamase, said method comprising the above steps of genotyping SHV beta lactamase, and additionally the step of: determining the presence or absence of an antibiotic resistance depending on the presence or absence of a SHV beta lactamase genotype associated with an antibiotic resistance.

Additionally, the present invention discloses an oligonucleotide array comprising at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe; said at least one first oligonucleotide capture probe being present on said at least one support in a first localized area, and said at least one second oligonucleotide capture probe being present on said at least one support in a second localized area.

Additional features and advantages of the present invention are described in, and will be apparent from, the following Detailed Description of the Invention and the figures.

### BRIEF DESCRIPTION OF THE FIGURES

FIG. 1 shows mean relative signal intensities of probe versions 1 and 2 of (a) probe No 234/5 hybridized with bla_{SHV-1} (n=3), (b) probe No 234/5 hybridized with bla_{SHV-2} (n=3), (c) probe No 234/5 hybridized with bla_{SH-4} (n=3), (d) probe No 222 hybridized with bla_{SHV-1} (n=3), (e) probe No 238 hybridized with bla_{SHV-1} (n=3), (f) probe No 234/5 hybridized with bla_{SHV-1} (n=3).

FIG. 2(a) shows a layout of mutation capture probes on the SHV microarray. All mutation specific capture probes were spotted in triplicate. The mutation position is indicated above each triplicate. For the SNP specific probes the nucleotide at the central or essentially central base is indicated on the left side of each row as A, G, C, or T. For probe No 234/5 the probes are designated by the targeted amino acids at position 234/5 (GE, SE, SK, AE, AK) and the nucleotide at the third codon position for amino acid 235 (GEa or GEg) as indicated in the legend. For position 136, 188/9, 163 one probe triplet (WT) matching the wildtype sequence (SHV-1) and one probe triplet (MT) matching the mutant sequence (SHV-X) are indicated. Negative and positive hybridization controls are indicated as neg. hyb. and pos. hyb controls respectively.

FIG. 2(b) shows a fluorescence image of a hybridization experiment with 200 ng target DNA bla_{SHV-1} (NT/F=55) for 1 h in an HS400 hybridization station. The signal intensity is shown in false color. In the original Figure (not shown), Blue corresponds to the lowest signal intensity, and red to white depict the highest signal intensities (as output by ImaGene software [version 3.0]).

FIG. 3(a) shows mean relative signal intensities for each SNP position and corresponding probe (A, G, C, and T refer to the base at the central or essentially central position), FIG. 3(b) shows mutation position and corresponding probe 234/5 designated by the targeted amino acids at position 234/5 (GE, SE, SK, AE, AK) and the nucleotide at the third codon position for amino acid 235 (GEa or GEg), and FIG. 3(c) shows mutation position and corresponding probe No 39, 136, 188/9, 163 WT matching the wildtype sequence (SHV-1) and MT matching the mutant sequence (SHV-X) from a hybridization experiment with 200 ng bla_{SHV} (NT/F=55) (n=3).

FIG. 4 shows relative (rel.) intensities of the signals for selected mutation positions according to the target DNA applied. The PMs at the mutation positions 4, 39, 175.1, 201, and 234/5 were identified to determine the origin of the target DNA: bla_{SHV-1} (n=3) (a), bla_{SHV-2} (n=3) (b), bla_{SHV-3} (n=3) (c), bla_{SHV-4} (n=3) (d), bla_{SHV-}5 (n=3) (e), bla_{SHV-7} (n=3) (f), bla_{SHV-8} (n=3) (g). For the SNP specific probes, the probe sets are indicated in the legend by their central base (A, G, C, or T), for probe 234/5 the probe set is designated by the targeted amino acids at position 234/5 (GE, SE, SK, AE, AK) and the nucleotide at the third codon position for amino acid 235 (GEa or GEg), for mutation position 39 WT is indicated in the legend for the probe matching the wildtype sequence (SHV-1) and MT for the probe matching the mutant sequence (SHV-X).

FIG. 5 shows Relative (rel.) intensities of the signals for mutation position 234/5 according to the target DNA applied: (a) pure 234/5 GEg containing target DNA, originating from three different isolates and five different amplification reactions, matching wildtype SHV-1. The five different target DNAs are indicated in the legend by a1 to a5 [a1 (n=1); a2 (n=3); a3 (n=3); a4 (n=1); a5 (n=1)] and (b) a mixture of 234/5 GEg (probably from chromosomally encoded SHV-1) in addition to 234/5 SKg containing target DNA originating from plasmid encoded SHV-5 from five different K. pneumoniae isolates. The five different target DNAs are indicated in the legend by b1 to b5 [b1 (n=2); b2 (n=2); b3 (n=2); b4 (n=2); b5 (n=1)].

### DETAILED DESCRIPTION OF THE INVENTION

The present invention provides a new approach for determining antibiotic resistances and in particular for detecting antibiotic resistances due to “Extended spectrum beta lactamases” (ESBLs) on the basis of a genotyping based detection of SHV beta lactamases, and in particular on the basis of a detection of bla_{SHV} gene sequences of SHV beta lactamase. Therefore, it is supposed that the present invention will provide an important help both to patients, as well as to persons working in the medical field, since Extended spectrum beta lactamases (ESBLs) are among the most important and problematic resistances to detect in bacteria, such as Escherichia coli and Klebsiella pneumoniae. They derive in particular from genes of the narrower-spectrum SHV-1 beta-lactamases by mutations that alter the amino acid configuration around the active site of these enzymes, causing increased resistance e.g. against oxyimino cephalosporins and monobactams.

The oligonucleotide micro-array according to the present invention allows a detection and discrimination of all 49 SHV beta-lactamase variants described to date which are related to the ESBL and/or IRT phenotype. The oligonucleotide array according to the present invention allows the discrimination of all currently 49 published SHV beta-lactamase sequences. By the use of a single consensus primer pair, the bla_{SHV} gene variants have been amplified and all mutation positions of the targeted genes can be analyzed simultaneously within 4 hours enabling the identification of the corresponding ESBL and/or IRT phenotype causing SHV beta-lactamase even in presence of a chromosomally encoded SHV-1 e.g. in Klebsiella pneumoniae.

Moreover, the array and method according to the present invention provide the possibility to specifically identify all gene variants, which are possibly causing resistance and thus deliver valuable epidemiologic information about the occurrence and distribution of different gene variants within a significantly reduced response time. Furthermore, for a number of ESBL variants a specific substrate pattern has already been characterized, which could be considered for an appropriate treatment choice, if the variant could be identified. So far this has only been achieved by sequencing the genes, which is technically demanding and time consuming.

The shortened analysis time (1-2 days for screening analysis, 4 hours for confirmation analysis) in comparison to standard phenotypic methods (more than 3 days) offers a potential health benefit and reduced treatment times for patients.

The terms “micro-array” and “oligonucleotide array” may be used interchangeably and refer to a multiplicity of different nucleotide sequences attached to one or more supports. Both terms may can refer to the entire collection of oligonucleotides on the support(s) or to a subset thereof.

The term “support” or “carrier” as used in the context of the present application refers to any material that provides a solid or semi-solid structure and a surface for attaching molecule(s). Such materials are preferably solid and include for example metal, glass, plastic, silicon, and ceramics as well as textured and porous materials. They also may include soft materials such as gels, rubbers, polymers, and other non-rigid materials. Preferred solid carriers are nylon membranes, epoxy-glass and borofluorate-glass. Solid carriers need not be flat and may include any type of shape including spherical shapes (e.g., beads or microspheres). Preferably solid carriers have a flat surface as for example in slides (such as object slides) and micro-titer plates, wherein a micro-titre plate is a dished container having at least two wells.

The expression “attached to a support” describes a non-random chemical or physical interaction by which a connection between two molecules is obtained. The attachment may be obtained by means of a covalent bond. However, the attachments need not be covalent or permanent. Other kinds of attachment include for example the formation of metalorganic or ionic bonds, binding based on van der Waal's forces, or any kind of enzyme substrate interactions or the so called affinity binding. An attachment to the surface of a carrier or carrier may also be referred to as immobilization. Each capture nucleotide may be connected to spacer molecules present at specifically localized areas on the surface of the carrier and may be attached by means of said spacers to the surface of the carrier. The localized area is either known by the construction of the microarray or is defined during or after the detection and results in a specific pattern.

As used in present invention, the term “probe” is defined as an oligonucleotide capable of binding to a target nucleic acid of complementary sequence through one or more types of chemical bonds, usually through complementary base pairing, usually through hydrogen bond formation. As used herein, an oligonucleotide capture probe may include natural (i.e. A, G, C, or T) or modified bases (7-deazaguanosine, inosine, etc.). In addition, the bases in oligonucleotide capture probe may be joined by a linkage other than a phosphodiester bond, such as e.g. peptide bonds, so long as it does not interfere with hybridization.

The term “target nucleic acid” refers to a nucleic acid, to which the oligonucleotide capture probe specifically hybridizes.

The term “capture probes” in the sense of the present invention shall designate parts of the beta-lactamase gene of different length, parts perfectly complementary thereto, as well as probes prepared according to the method for designing an oligonucleotide probe of the present invention, said capture probes having e.g. between about 10 and 43 nucleotides, which are either chemically synthesized in situ on the surface of the support or laid down thereon. A “capture probe” according to the present invention comprises a capture oligonucleotide sequence and optionally one or more adjacent moieties, such as e.g. a spacer moiety.

The term “perfect match probe” refers to a probe that has a sequence that is perfectly complementary to a particular target sequence.

The term “nucleotide sequence” as used herein refers to nucleotides present in nucleic acids compared with the bases of said nucleic acid, and includes nucleotides comprising usual or modified bases as above described. References to nucleotide(s), oligonucleotide(s), polynucleotide(s) and the like include analogous species wherein the sugar-phosphate backbone is modified and/or replaced, provided that its hybridization properties are not destroyed.

The phrase “hybridizing specifically to” refers to the binding, duplexing or hybridizing of a molecule substantially to or only to a particular nucleotide sequence or sequences under stringent conditions when that sequence is present in a complex mixture of DNA or fragments thereof.

The term “resistance mutation” as used herein relates to a mutation(s) of a gene which confer(s) to a bacterium, a resistance to a particular antibacterial compound, e.g. an antibiotic, so that the organism with the resistance mutation(s) may survive higher concentrations of said antibacterial compound than without said mutation(s). A resistance mutation may have the form of single nucleotide polymorphism and thus be detected using microarray technologies, in that at a specific position within the nucleotide sequence the bases have to be wobbled or altered according to the possibilities (at maximum four).

“Spacers” are molecules that have a first end attached to the probe and a second end attached to a carrier. Thus, the spacer molecule separates carrier and biological material, but is attached to both. The spacers may be synthesized directly or preferably attached as whole on the carrier at specific locations. Bindings within the spacer may include carbon-carbon single bonds, carbon-carbon double bonds, carbon-nitrogen single bonds, or carbon-oxygen single bonds. Spacers suitable for use in the present invention include e.g. nucleotides, which contain adenine, cytosine, guanine and thymine as bases and deoxyribose as the structural element. Furthermore, a nucleotide can, however, also comprise any artificial base known to current technology, which is capable of base pairing using at least one of the aforesaid bases (for example inosine). Preferred spacers comprise also thymidine spacers, which may have a variable length. The spacer may be also designed to minimize template independent noise, which is the result of signal detection independent (in the absence) of the template. The spacer further has suitable reactive groups, preferably at each end of for the attachment to the carrier, capture and probe molecule, respectively. Such reactive groups may comprise for example hydroxy-, thiol-, aldehyde-, amide- and thioamide-groups. In addition, the spacer may have side chains or other substitutions. The active group may be reacted by suitable means to form for example preferably a covalent bound between the spacer and carrier, capture or probe molecule. Suitable means comprise for example light. The reactive group may be optionally masked/protected initially by protecting groups. Among a wide variety of protecting groups, which are useful are for example FMOC, BOC, t-butyl esters, t-butyl ethers. The reactive group is used to attach specifically thereto (after the cleavage of the protecting group) another molecule.

An oligonucleotide may be produced according to any method known to the skilled person, including chemical synthesis, DNA replication, reverse transcription, PCR, or a combination thereof.

Hybridization is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, and the melting temperature of the formed hybrid. Hybridization involves the annealing of one nucleic acid to another complementary nucleic acid, i.e., a nucleic acid having a complementary nucleotide sequence.

“Stringency” refers to the conditions, which are involved in a correct hybridization event, for example temperature, ionic strength, pH and the presence of other compounds, under which nucleic acid hybridizations are conducted. The respective conditions can be easily chosen by a skilled person on the basis of his general knowledge in the art.

The term “sample” is meant to include a specimen or culture (for example microbiological cultures) and all kind of biological and environmental samples. Biological samples may be animal, including human, fluid, such as blood or urine, solid or tissue, alternatively food and feed products and ingredients such as dairy items, vegetables, meat and meat by-products. Environmental samples include environmental material such as surface matter, soil, water, industrial samples and waste, for example samples obtained from sewage plant, as well as samples obtained from food and dairy processing instruments, apparatus, equipment, utensils, disposable and non-disposable items.

The term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another.

The present invention provides a method for genotyping SHV beta lactamase by determining the presence or absence of at least one single nucleotide polymorphism (SNP), preferably at least five SNPs, more preferably at least ten SNPs, and especially of all so far known SNPs present in SHV beta lactamase genome. In a first step, a target DNA of SHV beta lactamase to be analyzed is provided. The target DNA may be isolated from a wide variety of sample material to be analyzed, such as e.g. body fluids, samples of bacteria, etc. according to standard procedures known to a skilled person.

Subsequently this target DNA of SHV beta lactamase will be amplified and labeled according to standard methods known in the state of the art.

The amplification may be performed according to any method known in the art, for example by using as amplification primers for the bla_{SHV} gene the forward primer “shvforw”: (5′-gcaaaacgccgggttattc-3′; SEQ ID NO: 99) and reverse primer “shvrev” (5′-ggttagcgttgcca gtgct-3′; SEQ ID NO: 100) and by using any known DNA polymerase, e.g. Taq DNA Polymerase.

As a “label” or marker any atom or molecule can be used which provides a detectable (preferably quantifiable) effect and which can be attached to a nucleic acid. The term “label” includes e.g. colored dyes; radioactive labels; binding moieties such as biotin; haptens such as digoxgenin; luminogenic, phosphorescent or fluorogenic moieties; and fluorescent dyes alone or in combination with moieties that can suppress or shift emission spectra by the energy transfer of fluorescence. Labels may provide signals, which are detectable for example by fluorescence, radioactivity, colorimetry, gravimetry, X-ray diffraction or absorption, magnetism and enzymatic activity. A label may be a charged moiety (positive or negative charge) or may also have a neutral charge. They may include or consist of nucleic acid or protein sequence. Preferred labels are dyes and in particular preferred labels are fluorescent dyes. The amplification and the labeling can be performed either simultaneously or in subsequent steps.

Under the aspect of efficiency, it is in particular advantageous to perform said labeling step simultaneously with said amplification step, e.g. by incorporating a dye labeled nucleotide, such as e.g. Cy3-dCTP into said amplified DNA.

The thus obtained amplified and labeled target DNA is then fragmented according to any standard method known to a skilled person into smaller fragments, in particular by a DNase, such as e.g. DNase I. The fragment size obtained can be determined by various methods known in the state of the art, such as e.g. lab-on-a-chip electrophoresis.

In particular, it has been shown that it is even possible to use a non-separated mixture of fragments directly during the following step, so that a step of isolating or separating these smaller fragments from the mixture of fragments obtained may be omitted. In particular good results were obtained, when said fragments of amplified and labeled target DNA have a size of 15 to about 150 bp (base pairs). If desired, specific fragments can be of course isolated according to standard methods from said mixture of fragments.

In a next step, said fragments are contacted with an array of oligonucleotide capture probes. This array of oligonucleotide capture probes has at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe. In particular, said oligonucleotide capture sequence capable of hybridizing specifically to a sequence of bla_{SHV} gene of SHV beta lactamase is an oligonucleotide sequence completely complementary to a sequence of bla_{SHV} gene of SHV beta lactamase or a modified oligonucleotide capture probe as outlined below.

The nucleotide of interest is a known position of a SNP in the bla_{SHV} gene of SHV beta lactamase. For each SNP a probe set of preferably 4 probes can be designed with identical sequence except the central base or an essentially centrally located base, which is either A, T, G, or C. The term essentially centrally means, that said nucleotide of interest is separated from the ends of said oligonucleotide capture sequence by at least one, preferably at least three, more preferably at least five nucleotides. When desired, instead of either A, T, G, or C, also a non-naturally occurring sequence can be incorporated. In case of an amino acid substitution position (e.g. 175) having two polymorphisms within one triplet, two SNP probe sets can be designed. In case, when in neighboring amino acid position (such as e.g. 234 and 235) multiple amino acid exchanges occur or when in amino acid positions more than one neighboring nucleotide differs in the mutant sequence from the wild type sequence, an approach as outlined in detail in the Examples can be applied.

Subsequently, the fragments of target DNA are subjected to a hybridizing reaction in order to obtain a hybridization between the target DNA and the oligonucleotide capture probes. These hybridizations can be carried out according to the general knowledge in the art by a skilled person.

Next, the presence of a hybridization of a target DNA fragment to said at least one first oligonucleotide probe will be determined and also the presence of a hybridization of a target DNA fragment to said at least one second oligonucleotide probe will be determined. While the hybridization to said first oligonucleotide capture probe is indicative for the presence of SHV beta lactamase without single nucleotide polymorphism at the position of said at least one nucleotide of interest, the hybridization to said second oligonucleotide capture probe being indicative for the presence of a SHV beta lactamase having said at least one single nucleotide polymorphism at the position of said at least one nucleotide of interest.

These determination steps can be performed e.g. essentially simultaneously. In the context of the present invention, the term “essentially simultaneous detection on a micro-array indicates that one or more target sequences may be screened for on a micro-array, the presence or absence thereof being determined essentially simultaneously depending on the specifically applied preparation and reading methods, etc.

The method of the present invention can be further elaborated by performing the before-mentioned steps, using instead of said at least one second oligonucleotide capture probe comprising an oligonucleotide capture sequence differing in at least one nucleotide of interest from said oligonucleotide capture sequence of said first oligonucleotide capture probe at least one third (fourth, fifth, sixth, . . . ) oligonucleotide capture probe comprising an oligonucleotide capture sequence differing in at least one nucleotide of interest from said oligonucleotide capture sequences of said first and second oligonucleotide capture probe. Preferably as many types of oligonucleotide capture probes will be provided that the discrimination of all currently 49 published SHV beta-lactamase sequences is possible.

Table A below shows the mutations and the corresponding SHV mutants: a Ambler position, position of the polymorphism in the amino acid sequence of SHV (http://www.lahey.org/ Studies/shvtable.asp) according to Ambler et al. (Ambler, R. P., A. F. W. Coulson, J. M. Frere, J. M. Ghuysen, B. Joris, M. Forsman, R. C. Levesque, G. Tiraby, and S. G. Waley. 1991. A Standard Numbering Scheme for the Class-A Beta-Lactamases. Biochemical Journal 276:269-270); position, position of the mutation according to SHV amino acid sequence. The amino acids in the SHV-1 sequence and the mutated SHV (SHV-X) sequence are also indicated. b The numbers in the table body are the SHV types with an amino acid substitution at the indicated position. The ESBL and IRT phenotypes are as described by Bradford (Bradford, P. A. 2001. Extended-spectrum beta-lactamases in the 21st century: characterization, epidemiology, and detection of this important resistance threat. Clin. Microbiol. Rev. 14:933-51, table). ^{c} Position refers to the position of the mutation in the nucleotide sequence of the bla_{SHV} gene (GenBank accession number AF462396).

TABLE A
SHV beta lactamase polymorphism sites and corresponding mutants

| | | |
|---|---|---|
| Nucleotide | Amino Acid^{a} | SHV-X with the following phenotype^{b} |

| | | | |
|---|---|---|---|
| Codon in: | Ambler | Amino acid in: | not |

| | | | | | | | | |
|---|---|---|---|---|---|---|---|---|
| blaSHV-1 | blaSHV-X | position | Position | SHV-1 | SHV-X | ESBL | IRT | defined |

| | | | | | | | | | |
|---|---|---|---|---|---|---|---|---|---|
| 8 | TAT | TTT | 7 | 3 | Y | F | | | 28 |
| 10 | ATT | TTT | 8 | 4 | I | F | 7, 14, 18, 34 | | |
| 10 | ATT | GTT | 8 | 4 | I | V | | | 36 |
| 40 | ACC | GCC | 18 | 14 | T | A | | | 25, 37 |
| 61 | GCC | TCC | 25 | 21 | A | S | | | 42 |
| 92 | CTA | CAA | 35 | 31 | L | Q | 2a, 12, 13, 15, | | 11, 25, 35, 36, |
| | | | | | | | 29 | | 37, 40 |
| 115 | CGC | AGC | 43 | 39 | R | S | 7, 14, 18, 29, | | |
| | | | | | | | 34 | | |
| 130 | GAA | AAA | 48 | 44 | E | K | 15 | | |
| 148- | GGC | — | 54 | 50 | G | Del | 9 | 10 | |
| 150 | | | | | | | | | |
| 180 | GAA | GGA | 64 | 60 | E | G | 34 | | |
| 226 | GTG | ATG | 80 | 76 | V | M | 15 | | |
| 253 | GAA | AAA | 89 | 85 | E | K | 15 | | 35 |
| 325 | CTT | TTT | 113 | 109 | L | F | | | 43 |
| 343 | GTC | ATC | 119 | 115 | V | I | 48 | | |
| 352 | CTC | TTC | 122 | 118 | L | F | 21 | | |
| 365 | GCC | GTC | 126 | 122 | A | V | | | 32 |
| 373 | ATG | GTG | 129 | 125 | M | V | 25 | | 37, 42 |
| 376 | AGC | GGC | 130 | 126 | S | G | | 10 | |
| 406- | GCC | CGG | 140 | 136 | A | R | 9 | 10 | |
| 408 | | | | | | | | | |
| 412 | GTC | TTC | 142 | 138 | V | F | | | 41 |
| 422 | CCC | CTC | 145 | 141 | P | L | | | 51 |
| 425 | GCA | GTA | 146 | 142 | A | V | | | 38 |
| 433 | ACT | TCT | 149 | 145 | T | S | | | 43 |
| 455 | GGC | GAC | 156 | 152 | G | D | | | 27, 32, 45 |
| 462 | AAC | AAG | 158 | 154 | N | K | 22 | | |
| 474- | | TGA . . . AC | 167- | 163- | | DRWET | | | 16 |
| 475 | | insert | 168 | 164 | | insert | | | |
| 505 | CTT | TTT | 173 | 169 | L | F | 19, 20, 21 | | |
| 512 | GGC | GCC | 175 | 171 | G | A | | | 51 |
| 523 | GAC | AAC | 179 | 175 | D | N | 8 | | |
| 524 | GAC | GCC | 179 | 175 | D | A | 6 | | |
| 524 | GAC | GGC | 179 | 175 | D | G | 24 | | |
| 547 | GCC | ACC | 187 | 183 | A | T | 26, | | 50 |
| 564 | AAG | AAC | 192 | 188 | K | N | 9, 10 | | |
| 565 | CTG | GTG | 193 | 189 | L | V | 9, 10 | | |
| 572 | ACC | AAC | 195 | 191 | T | N | 46 | | |
| 572 | ACC | ATC | 195 | 191 | T | I | | | 53 |
| 602 | CGG | CTG | 205 | 201 | R | L | 3, 4, | | 44 |
| 606 | CAG | CAT | 206 | 202 | Q | H | | | 50 |
| 664 | CCG | TCG | 226 | 222 | P | S | | | 33 |
| 700- | GGCGAG/A | AGCGAA/G | 238/ | 234/ | GEg | SE | 2, 2a, 3, 20, 21, | | |
| 705 | | | 240 | 235 | | | 30, 34, 39 | | |
| 700- | GGCGAG/A | AGCAAA/G | 238/ | 234/ | GEg | SK | 4, 5, 7, 9, 12, | 10 | |
| 705 | | | 240 | 235 | | | 15, 22, 45, 46, | | |
| | | | | | | | 47, 48 | | |
| 700- | GGCGAG/A | GCCGAA/G | 238/ | 234/ | GEg | AE | 29, 13 | | |
| 705 | | | 240 | 235 | | | | | |
| 700- | GGCGAG/A | GCCAAA | 238/ | 234/ | GEg | AK | 18 | | |
| 705 | | | 240 | 235 | | | | | |
| 713 | GCG | GGG | 243 | 238 | A | G | | | 35, 40 |
| 784 | AGG/G | TCC | 267 | 262 | T | S | 39 | | |
| 816 | CAA | CAT | 278 | 272 | Q | H | | | 37 |

In particular, the method of the present invention may be used for e.g. genotyping SHV beta lactamase of Klebsiella pneumoniae or Escherichia coli and especially of Klebsiella pneumoniae having a beta lactamase bearing a chromosomally encoded SHV.

In order to provide more information, the present method for genotyping SHV beta lactamase may be combined with an identification of the presence of one or more TEM beta lactamase genotypes (Grimm, V., S. Ezaki, M. Susa, C. Knabbe, R. D. Schmid, and T. T. Bachmann. 2004. Use of DNA microarrays for rapid genotyping of TEM beta-lactamases that confer resistance. J. Clin. Microbiol. 42:3766-3774. The content of this publication is herewith incorporated by reference). Surprisingly, both assay systems showed up to have operation conditions permitting a combination. In combination with the TEM microarray this system is capable to identify the majority of the clinically relevant ESBL-type enzymes.

The present invention also provides a method of antibiotic resistance determination. The method comprises in addition to steps of genotyping SHV beta lactamase as above also a step of determining the presence or absence of an antibiotic resistance depending on the presence or absence of a SHV beta lactamase genotype associated with an antibiotic resistance.

During the extensive studies leading to the present invention, the inventors established that for several specific amino acid positions, i.e. 39, 222, 234/5, 238, and 262, the calculated dimer dG resulting for the first probe versions, which were designed (see dG values as indicated in Table 1) were unfavorable. Since the array of the present invention should also include the relevant mutation positions for unequivocal identification of all naturally occurring SHV variants, the present invention discloses a method surprisingly permitting also a reliable identification of amino acid positions which do not provide a sufficient specific hybridization to a oligonucleotide capture sequence having a complementary sequence. Without wishing to be bound to any theory, it can be assumed that this insufficient specific hybridization is due to dimer forming bases in close proximity to the mutation position. Therefore, the present inventors developed a new probe design concept starting from the finding that through introduction of an additional mismatch the secondary structure can be dissolved. Surprisingly, the oligonucleotide capture sequence retained a sufficient hybridization capability to allow a discrimination between mismatched base and the perfect match base at the SNP position to be identified.

This method for designing an oligonucleotide probe comprises determining an oligonucleotide capture probe completely complementary to a sequence of bla_{SHV} gene of a SHV beta lactamase and having an insufficient hybridizing capability with respect to a perfect matching sequence, designing an adapted oligonucleotide by replacing at least one nucleotide by another naturally occurring nucleotide or a nucleotide equivalent. When necessary, the discrimination capability may be even improved by adjusting the probe positions and length for optimal accessibility of the new probes. The term nucleotide equivalent comprises any modified base, such as e.g. 7-deazaguanosine, inosine, etc., known to a skilled person. In particular, the length of the oligonucleotide capture sequence can be adapted by deleting one or more nucleotides present in terminal position in the oligonucleotide capture sequence or by adding one or more nucleotides at terminal position.

According to another aspect, the present invention provides isolated oligonucleotides having a sequence as identified in SEQ. ID. NO. 1 to 46 and 47 to 94 which are indicated in the tables.

Table 1 shows probe secondary structure dG values, length, and sequences of oligonucleotide probes version1 (V1) and version2 (V2) of probe No 222, 234/5, 238 and 262; of probe No 39 only one version is displayed. Hairpin and Dimer dG values were calculated with default parameters for reaction conditions by Arraydesigner software (Premier Biosoft International, Palo Alto, Calif.). The exchanged nucleotide from probe version 1 to 2 is underlined. Table 2 below shows that for the SNP specific probes, four probes for each SNP position were used. The probes are named for the position in the amino acid sequence of bla_{SHV}. The four probes had either A, G, C, or T at the central or essentially central base position (designated N in the probe sequence). The probe length (between 14 and 24 bases) and the melting temperatures (calculated for the probes matching the SHV-1 sequence; DNA concentration 50 nM; salt conc, 50 mM by Arraydesigner software (Premier Biosoft International, Palo Alto, Calif.) are also provided.

TABLE 1

Oligonucleotide probe sequences
version 1 and version 2

| | | | | | |
|---|---|---|---|---|---|
| | | | Hair- | | SEQ. |
| | | | pin | Dimer | ID. |
| Name | Probe Sequence | Length | dG | dG | NO |

| | | | | | |
|---|---|---|---|---|---|
| 39°_V1 | TGTCGGGCAGCGTAGG | 16 | 0 | 0 | 1 |
| 39G_V1 | TGTCGGGCGGCGTAGG | 16 | 0 | 0 | 2 |
| 39G_V1 | TGTCGGGCCGCGTAGG | 16 | 0 | −5.1 | 3 |
| 39T_V1 | TGTCGGGCTGCGTAGG | 16 | 0 | 0 | 4 |
| 222A_V1 | CGTGCTGACGGCGGG | 15 | 0 | 0 | 5 |
| 222G_V1 | CGTGCTGGCGGCGGG | 15 | 0 | 0 | 6 |
| 222C_V1 | CGTGCTGCCGGCGGG | 15 | 0 | −8.9 | 7 |
| 222T_V1 | CGTGCTGTCGGCGGG | 15 | 0 | 0 | 8 |
| 222A_V2 | CCGTGCTGACTGCG | 14 | 0 | 0 | 9 |
| 222G_V2 | CCGTGGTGGCTGCG | 14 | 0 | 0 | 10 |
| 222C_V2 | CCGTGCTGCCTGCG | 14 | 0 | 0 | 11 |
| 222T_V2 | CCGTGCTGTCTGCG | 14 | 0 | 0 | 12 |
| 234/ | GGAGCTGGCGAACGGG | 16 | 0 | −2.9 | 13 |
| 5GEa_V1 | | | | | |
| 234/ | GGAGCTGGCGAGCGGG | 16 | −1.7 | −2.9 | 14 |
| 5GEg_V1 | | | | | |
| 234/ | GGAGCTAGCGAACGGG | 16 | 0 | −6.2 | 15 |
| 5SEa_V1 | | | | | |
| 234/ | GGAGCTAGCGAGCGGG | 16 | −1.7 | −6.2 | 16 |
| 5SEg_V1 | | | | | |
| 234/ | GGAGCTAGCAAACGGG | 16 | 0 | −6.2 | 17 |
| 5SKa_V1 | | | | | |
| 234/ | GGAGCTAGCAAGCGGG | 16 | −1.7 | −6.2 | 18 |
| 5SKg_V1 | | | | | |
| 234/ | GGAGCTGCCGAACGGG | 16 | 0 | −2.9 | 19 |
| 5AEa_V1 | | | | | |
| 234/ | GGAGCTGCCGAGCGGG | 16 | −1.7 | −2.9 | 20 |
| 5AEg_V1 | | | | | |
| 234/ | GGAGCTGCCAAACGGG | 16 | 0 | −2.9 | 21 |
| 5AK_V1 | | | | | |
| 234/ | GGAGCTGGTGAACGG | 15 | 0 | −2.9 | 22 |
| 5GEa_V2 | | | | | |
| 234/ | GGAGCTGGTGAGCGG | 15 | −1.7 | −2.9 | 23 |
| 5GEg_V2 | | | | | |
| 234/ | GGAGCTAGTGAACGG | 15 | 0 | −2.9 | 24 |
| 5SEa_V2 | | | | | |
| 234/ | GGAGCTAGTGAGCGG | 15 | −1.7 | −2.9 | 25 |
| 5SEg_V2 | | | | | |
| 234/ | GGAGCTAGTAAACGG | 15 | 0 | −2.9 | 26 |
| 5SKa_V2 | | | | | |
| 234/ | GGAGCTAGTAAGCGG | 15 | −1.7 | −2.9 | 27 |
| 5SKg_V2 | | | | | |
| 234/ | GGAGCTGCTGAACGG | 15 | 0 | −2.9 | 28 |
| 5AEa_V2 | | | | | |
| 234/ | GGAGCTGCTGAGCGG | 15 | −1.7 | −2.9 | 29 |
| 5AEg_V2 | | | | | |
| 234/ | GGAGCTGCTAAACGG | 15 | 0 | −2.9 | 30 |
| 5AK_V2 | | | | | |
| 238A_V1 | GGGGTGAGCGCGGGAT | 16 | 0 | −5.1 | 31 |
| 238G_V1 | GGGGTGGGCGCGGGAT | 16 | 0 | −5.1 | 32 |
| 238T_V1 | GGGGTGTGCGCGGGAT | 16 | 0 | −5.1 | 33 |
| 238C_V1 | GGGGTGCGCGCGGGAT | 16 | 0 | −9.8 | 34 |
| 238A_V2 | CGGGGTGAGTGCGGG | 15 | 0 | 0 | 35 |
| 238G_V2 | CGGGGTGGGTGCGGG | 15 | 0 | 0 | 36 |
| 238C_V2 | CGGGGTGCGTGCGGG | 15 | 0 | 0 | 37 |
| 238T_V2 | CGGGGTGTGTGCGGG | 15 | 0 | 0 | 38 |
| 262A_V1 | GGGATACCCCGGCGA | 15 | 0 | −4.2 | 39 |
| 262G_V1 | GGGATGCCCCGGCGA | 15 | 0 | −4.2 | 40 |
| 262C_V1 | GGGATGCCCCGGCGA | 15 | 0 | −9.5 | 41 |
| 262T_V1 | GGGATTCCCCGGCGA | 15 | 0 | −4.2 | 42 |
| 262A_V2 | CTGCGTGATAGCCCG | 15 | 0 | 0 | 43 |
| 262G_V2 | CTGCGTGATGCCCCG | 15 | 0 | 0 | 44 |
| 262C_V2 | CTGCGTGATCGCCCG | 15 | 0 | −1.9 | 45 |
| 262T_V2 | CTGCGTGATTCCCCG | 15 | 0 | 0 | 46 |

TABLE 2

Oligonucleotide probe sequences used
in the SHV-specific microarray

| | | | | |
|---|---|---|---|---|
| Melting | | | | SEQ. |
| temp | Length | | | ID. |
| (° C.) | (bases) | Name | Probe sequence (5′-3′) | NO. |

| | | | | |
|---|---|---|---|---|
| 50.2 | 19 | 3 | ATGCGTTNTATTCGCCTGT | 47 |
| 50.2 | 19 | 4 | ATGCGTTATNTTCGCCTGT | 48 |
| 55.5 | 18 | 14 | CTGTTAGCCNCCCTGCCG | 49 |
| 54.6 | 16 | 21 | CGGTACACNCCAGCCC | 50 |
| 55.4 | 24 | 31 | AGCAAATTAAACNAAGCGAAAGCC | 51 |
| 56.0 | 16 | 39 | TGTCGGGCNGCGTAGG | 52 |
| 55.8 | 25 | 44 | GTAGGCATGATANAAATGGATCTGG | 53 |
| 54.9 | 18 | 60 | CGCCGATGNACGCTTTCC | 54 |
| 55.9 | 15 | 76 | CGGCGCANTGCTGGC | 55 |
| 54.8 | 17 | 85 | GCCGGTGACNAACAGCT | 56 |
| 54.3 | 21 | 109 | TCGAAAAACACNTTGCCGACG | 57 |
| 55.4 | 17 | 115 | GCATGACGNTCGGCGAA | 58 |
| 52.7 | 16 | 118 | GTCGGCGAANTCTGCG | 59 |
| 56.0 | 16 | 122 | CGCCGCCGNCATTACC | 60 |
| 58.8 | 25 | 125 | CCGCCATTACCNTTAGCGATAACAG | 61 |
| 56.2 | 23 | 126 | CCATTACCATGNGCGATAACAGC | 62 |
| 60.9 | 20 | 136WT | CTGCTACTGGCCACCGTCGG | 63 |
| 60.1 | 20 | 136MT | CTGCTACTCCGGACCGTCGG | 64 |
| 57.4 | 15 | 138 | GGCCACCNTCGGCGG | 65 |
| 56.1 | 15 | 141 | GCGGCCNCGCAGGAT | 66 |
| 55.0 | 16 | 142 | CGGCCCCGNAGGATTG | 67 |
| 43.1 | 16 | 145 | AGGATTGNCTGCCTTT | 68 |
| 53.2 | 17 | 152 | CCAGATCGNCGACAACG | 69 |
| 56.1 | 17 | 154 | CGGCGACAANGTCACCC | 70 |
| 52.4 | 20 | 163WT | CTGGGAAACGGAACTGAATG | 71 |
| 58.7 | 20 | 163MT | CTGGGAAACTGACCGCTGGG | 72 |
| 53.4 | 16 | 169 | ATGAGGCGNTTCCCGG | 73 |
| 55.1 | 15 | 175.1 | CGCCCGCNACACCAC | 74 |
| 53.2 | 15 | 175.2 | GCCCGGGNCACCACT | 75 |
| 55.9 | 16 | 183 | CCAGCATGNCCGCGAC | 76 |
| 55.9 | 17 | 188/9WT | GCGCAAGCTGCTGACCA | 77 |
| 56.3 | 17 | 188/9MT | GCGCAACGTGCTGACCA | 78 |
| 56.0 | 16 | 191 | GCTGCTGANCAGCCAGC | 79 |
| 54.8 | 17 | 201 | GTTCGCAACNGCAGCTG | 80 |
| 54.0 | 16 | 202 | GCAACGGCANCTGCTG | 81 |
| 51.5 | 14 | 222 | CCGTGCTGNCTGCG | 82 |
| 50.2 | 15 | 234/5 GEg | GGAGCTGGTGAGCGG | 83 |
| 47.4 | 15 | 234/5 GEa | GGAGCTGGTGAACGG | 84 |
| 43.6 | 15 | 234/5 SEa | GGAGCTAGTGAACGG | 85 |
| 46.4 | 15 | 234/5 SEg | GGAGCTAGTGAGCGG | 86 |
| 40.4 | 15 | 234/5 SKa | GGAGCTAGTAAACGG | 87 |
| 43.3 | 15 | 234/5 SKg | GGAGCTAGTAAGCGG | 88 |
| 48 | 15 | 234/5 AEa | GGAGCTGCTGAACGG | 89 |
| 50.8 | 15 | 234/5 AEg | GGAGCTGCTGAGCGG | 90 |
| 44.8 | 15 | 234/5 AK | GGAGCTGCTAAACGG | 91 |
| 57.7 | 15 | 238 | CGGGGTGNGTGCGGG | 92 |
| 47.3 | 15 | 262 | CTGCGTGATNCCCGG | 93 |
| 54.6 | 19 | 272 | AAATCAGCANATCGCCGGG | 94 |
| 54.9 | 21 | process | TTTAAAGTAGTGCTCTGCGGC | 95 |
| | | control | | |
| 46.0 | 18 | negative | TCTAGACAGCCACTCATA | 96 |
| | | hybridi- | | |
| | | zation | | |
| | | control | | |
| 53.1 | 19 | positive | GATTGGACGAGTCAGGAGC | 97 |
| | | hybridi- | | |
| | | zation | | |
| | | control | | |
| 46.0 | 18 | spot | TCTAGAGAGCGACTGATA-Cy3 | 98 |
| | | control | | |

The probes No 234/5 are designated by the targeted amino acids at position 234/5 (GE, SE, SK, AE, AK) and the nucleotide at the third codon position for amino acid 235 (e.g. GEa or GEg). For probes where more than one neighboring nucleotide differs in the mutant sequence from the wildtype sequence, one probe (136WT, 188/9WT, 163WT) matching the wildtype sequence (SHV-1) and one probe (136MT, 188/9MT, 163MT) matching the mutant sequence were designed. The triplet with the amino acid substitution is underlined. All probes carried a 13T spacer and a C6-amino modification at the 3′end.

Moreover, the present invention provides also molecules comprising a sequence as identified in SEQ. ID. NO. 1 to 94, and at least one spacer moiety as defined above attached to at least one end of said oligonucleotide.

These oligonucleotides have a sequence as identified in SEQ. ID. NO. 1 to 94 and the molecules comprising a sequence as identified in SEQ. ID. NO. 1 to 94 can be used as capture probes.

The present invention provides also an oligonucleotide array comprising at least one first oligonucleotide capture probe comprising an oligonucleotide capture sequence capable of hybridizing specifically to a sequence of bla_{SHV} gene of a SHV beta lactamase and at least one second oligonucleotide capture probe comprising an oligonucleotide capture sequence differing in at least one nucleotide of interest from said oligonucleotide capture sequence of said first oligonucleotide capture probe; said at least one first oligonucleotide capture probe being present on said at least one support in a first localized area, and said at least one second oligonucleotide capture probe being present on said at least one support in a second localized area.

For example, in an oligonucleotide array according to the present invention said at least one second oligonucleotide capture probe comprises a capture sequence selected from the group consisting of sequences having SEQ ID NO: 1 to 94. Preferably at least five different further oligonucleotide probes comprising each a different sequence selected from the group consisting of sequences having SEQ ID NO: 1 to 94, more preferably at least ten different further oligonucleotide probes comprising each a different sequence selected from the group consisting of sequences having SEQ ID NO: 1 to 94 will be comprised on said array, each oligonucleotide capture probe on a respective localized area. Advantageously, as many types of oligonucleotide capture probes will be provided that the discrimination of all currently 49 published SHV beta-lactamase sequences is possible.

The support can be made of any material known as an array support material to a skilled person, for example, the materials as detailed above. In particular, the support can be a slide, preferably an epoxy coated glass slide.

In addition, the oligonucleotide array can include at least one of the following controls selected from the group consisting of a spotting control, a positive hybridization control, a negative hybridization control and a process control. Spotting control, positive hybridization control and negative hybridization control consist of sequences unrelated to bacterial species, whereas a process control corresponds to a conserved sequence within the bla_{SHV} gene family.

In particular, the oligonucleotide microarray according to the present invention permits a detection and discrimination of all 49 SHV beta-lactamase variants described to date which are related to the ESBL and/or IRT phenotype (see http://www.lahey.org/Studies/webt. asp#SHV). By the use of a single consensus primer pair, the bla_{SHV} gene variants could be amplified and all mutation positions of the targeted genes could be analyzed simultaneously within 4 hours enabling the identification of the corresponding ESBL and/or IRT phenotype causing SHV beta-lactamase even in presence of a chromosomally encoded SHV-1, e.g. in Klebsiella pneumoniae. In combination with the TEM microarray this system is capable to identify the majority of the clinically relevant ESBL-type enzymes.

The SHV microarray was tested with target DNA, originating from E. coli and K. pneumoniae. The target DNA was amplified and fluorescently labeled by PCR using consensus primers in presence of Cy3-labeled nucleotides. The total assay including PCR, hybridization, and image analysis could be performed in 4 hours. The identified variants included SHV-1, -2, -3, -4, -5, -7, and SHV-8. Also mixed resistances such as an ESBL variant (SHV-5) in presence of a narrower-spectrum variant (SHV-1) could be detected in the K. pneumoniae isolates. The microarray results were validated by standard clinical procedures. Here, the microarrays outperformed in terms of assay time and information depth. In conclusion, these arrays offer an attractive option for the identification and epidemiologic monitoring of SHV beta lactamases within the clinical routine diagnostics.

The present invention provides also a kit comprising the oligonucleotide array according to the present invention and at least one of reaction reagent, preferably oligonucleotide(s), enzyme(s), and supporting material, preferably buffer(s), solvent(s) and instruction leaflet for carrying out an assay according to the present invention.

Moreover, the present invention discloses also amplification primers for amplifying the bla_{SHV} gene, said amplification primers having nucleotide sequences as identified in SEQ.ID. NO. 99 to 100.

The following examples illustrate the present invention and should not be construed to limit the scope of the present invention.

### EXAMPLES

### Example 1

Probe Design

For 37 mutation positions (ESBL, IRT, Tables A, 2) oligonucleotide probes were designed with variable lengths (15-25 bases) (see Table 1). Secondary structure's dG values were estimated with the Arraydesigner 2.0 software (Premier Biosoft International, Palo Alto, Calif.). The probes were designed with the mutation at the central or essentially central position within the probe sequence for maximum perfect match/mismatch discrimination during hybridization. For each SNP a probe set of 4 probes was designed with identical sequence except the central or essentially central base, which is either A, T, G, or C. The probes are named for the position of the amino acid substitution within the SHV sequence, that means probe No 03 defines the polymorphism at position 03 in the SHV amino acid sequence. For one amino acid substitution position (175) there are two polymorphisms within one triplet. For this two SNP probe sets (2×4 probes) were designed (e.g. 175.1, 175.1). For the neighboring amino acid position 234 and 235 multiple amino acid exchanges occur. For each naturally occurring SHV-mutant sequence one perfect match probe was designed. These probes are designated by the targeted amino acids at position 234/5 (GE, SE, SK, AE, AK) and the nucleotide at the third codon position for amino acid 235 (GEa or GEg). At three amino acid positions more than one neighboring nucleotide differs in the mutant sequence from the wildtype sequence e.g. (for amino acid position 135/6: GGCC to CCGG, 188/9: GC to CG, 163: insertion of 15 nucleotides), here for each position one probe (136WT, 188/9WT, 163WT) matching the wildtype sequence (SHV-1) and one probe (136MT, 188/9MT, 163MT) matching the mutant sequence were designed.

### Example 2

Oligonucleotide Array Fabrication

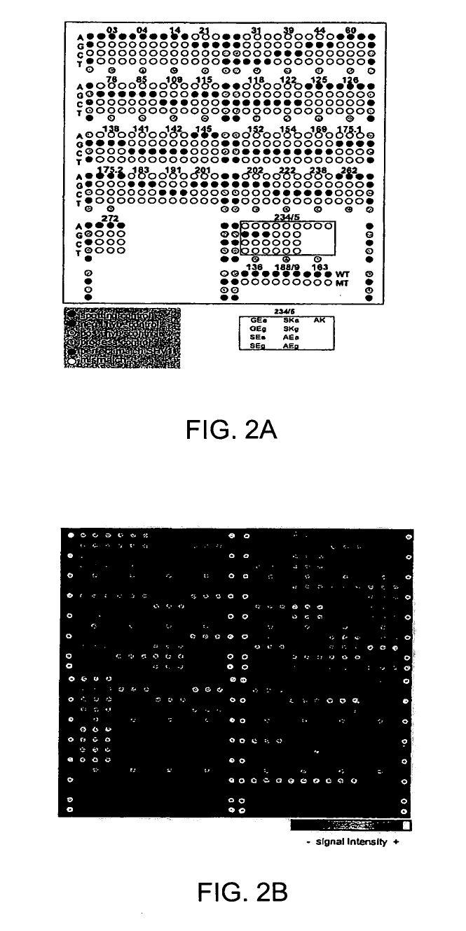

Oligonucleotide arrays were constructed with 151 oligonucleotide capture probes. The oligonucleotides were purchased from Metabion (München, Germany) at a desalted purity grade. The capture and control probe sequences are given in Table 1, all probes were equipped with a 13 thymidine spacer and a C6-amino-modification. Each mutation probe was spotted in triplicate. The array layout is shown in FIG. 2. The probes were dissolved in spotting buffer (160 mM Na₂SO₄, 130 mM Na₂HPO₄) to a final concentration of 20 or 40 μM (probe No. 234/5) and spotted with a MicroGrid II using MicroSpot 2500 pins (BioRobotics, Cambridge, UK) on epoxy coated glass slides (Elipsa AG, Berlin, Germany). For covalent immobilization the oligonucleotide array was incubated at 120° C. for 30 min in a drying compartment (Memmert, Schwabach, Germany). For blocking, the slides were rinsed for 5 min in 0.1% (v/v) Triton×100 in ddH2O, 4 min in 0.5 μl conc. HCl per ml ddH2O, for 10 min in 100 mM KCl solution while constantly stirring. Subsequently, the slides were incubated in blocking solution (25% (v/v) ethylenglycol, 0.5 μl conc. HCl per ml ddH2O) with the spotted side upwards at 50° C. in a heating compartment (OV5, Biometra, Göttingen, Germany). For cleaning, the slides were rinsed in ddH₂O for 1 min and then dried by nitrogen flow. The spot size was estimated to be 150 μm and the spot to spot distance 320 μm. Processed slides were stored for maximally 20 days dry at room temperature in the dark until further use.

Controls

Several controls were included on the array: a spotting control (5′-Cy3-ttttttttttttttctagacagcc actcata-3′), a positive hybridization control (5′-tttttttttttttgattggacgagtcaggagc-3′) complementary to a labeled oligonucleotide target (5′-Cy3-gctcctgactcgtccaatc-3′), which was spiked in during hybridization and a negative hybridization control (5′-ttttttttttttttctagacagcc actcata-3′). All these controls consisted of sequences unrelated to bacterial species. The process control (5′-ttttttttttttttttaaagtagtgctctg cggc-3′) corresponded to a conserved sequence within the bla_{SHV} gene family. The spotting controls were set at the corner positions of each subgrid, which was spotted by a different pin. The positive and negative hybridization controls appeared alternately at the side borders of each subgrid. The process controls were spotted in two lines bordering the central SNP probe sets of each subgrid (FIG. 2a).

### Example 3

Antibiotic Resistance Determination

Bacterial Strains

As reference samples E. coli DH5α transformed with bla_{SHV} target genes in a pCCR9 target vector were used, which were kindly provided by Herbert Hachler (Randegger, C. C., A. Keller, M. Irla, A. Wada, and H. Hachler. 2000. Contribution of natural amino acid substitutions in SHV extended-spectrum beta-lactamases to resistance against various beta-lactams. Antimicrobial Agents and Chemotherapy 44:2759-2763). The bacterial strains were inoculated in 5 ml LB-medium with 50 μg ampicillin per ml and incubated overnight at 37° C. Clinical samples were taken during the daily routine of the General Hospital Zadar (Zadar, Croatia) and processed following standard operation procedures. Bacterial strain identification and phenotypic antibiotic resistance determination was performed according to NCCLS standard operation procedures at the Robert Bosch Hospital in Stuttgart (NCCLS. 2001. Performance standards for antimicrobial susceptibility testing, eleventh informational supplement M100-S11. NCCLS, Wayne, Pa.). Prior microarray analysis, the bacterial strains were inoculated on Müller-Hinton agar plates at 37° C. over night. Plasmid DNA was extracted according to the QIAprep® Spin Miniprep Kit protocol (Qiagen, Hilden, Germany).

Amplification, Labeling and Purification of bla_{SHV} Target DNA

The target DNA for hybridization on the oligonucleotide arrays was synthesized by PCR (polymerase chain reaction). The sequence of the amplification primers for the bla_{SHV} gene (with an expected amplicon length of 932 bp) were for the forward primer “shvforw”: (5′-gcaaaacgccgggttattc-3′) and reverse “shvrev” (5′-ggttagcgttgccagtgct-3′). For PCR amplification and labeling 30-80 ng of plasmid DNA was supplemented with 0.4 μM forward and reverse primer, PCR-buffer (2% DMSO, 2.5 mM Mg(OAc)₂, 50 mM KCl, 10 mM Tris-HCl pH 8.3) containing 50 μM dATP, dGTP, dTTP, 30 μM dCTP, 20 μM Cy3-dCTP (Amersham Biosciences, Freiburg, Germany) and 10 U Taq DNA Polymerase (Eppendorf AG, Hamburg, Germany) in a total volume of 100 μl. The amplification was performed in a Mastercycler Gradient® (Eppendorf AG, Hamburg, Germany). An initial denaturation step (94° C. for 1 min) was followed by 30 cycles (94° C. for 1 min, 54° C. for 1 min, 72° C. for 1 min) and a final extension step at 72° C. for 4 min. The PCR product was purified with the Qiaquick® Spin PCR Purification Kit (Qiagen, Hilden, Germany) according to the manufactor's protocol. The DNA was eluted in 30 μl ddH₂O. The incorporation rate of Cy3-dCTP, expressed as number of nucleotides/incorporated fluorescent dye (NT/F) was determined by OD measurement (ND-1000 Spectrophotometer, NanoDrop Technologies, Rockland, USA).

Fragmentation

The amplified and labeled target DNA was diluted to a concentration of 30 ng/μl in reaction buffer (40 mM Tris-HCl, pH 8, 10 mM MgSO₄, 1 mM CaCl₂) and fragmented with DNase I (11.5 mU/μl) (Promega, Mannheim, Germany) at room temperature for 5 min to fragment sizes of about 15 to 150 bp in order to increase the efficiency of hybridization. The reaction was stopped by the addition of 3 mM EGTA and incubation at 65° C. for 10 min. Fragment sizes were estimated by lab-on-a-chip electrophoresis (Bioanalyzer 2100 and DNA 500 LabChip kit, Agilent, Boblingen, Germany).

Hybridization

Hybridizations were carried out with the hybridization station HS 400 (Tecan, Crailsheim, Germany) with an initial wash step at 45° C. with 6×SSPE for 30 s, followed by the probe injection (200 ng fragmented target DNA with incorporation ratios varying from 50-150 NT/F, 0.05 pmol control DNA (Cy3-gctcctgactcgtccaatc) in 65 μl 6×SSPE) and hybridization under medium agitation intensity for 1 hour at 45° C. The slides were then washed at room temperature for 2 times 2 min in 2×SSC (sodium salt citrate), 0.1% SDS (sodium dodecyl sulphate) and for 1.5 min in 0.2×SSC. The slides were dried with N₂ for 2 min.

Data Acquisition and Processing

Data from oligonucleotide arrays after the hybridization reaction were extracted by acquisition of fluorescence signals with a 418 Array Scanner (Affymetrix, Santa Clara, USA) at laser power and gain settings to 100%. The image processing and calculation of signal intensities was performed using Imagene™, Version 3.0 (Biodiscovery Inc., Los Angeles, USA). For the calculation of the individual net signal intensities the local background was subtracted from the raw spot intensity. To calculate the mean net signal intensity of one oligo probe on n arrays, n times three replicates of one spot were used, herein referred to as “signal intensity” (I). Within each oligo set related to one SNP, the highest signal intensity was taken as potential perfect match (PM). The remaining three oligo probes having a lower signal were considered as mismatch (MM). For comparison of inter array variation, mean intensity ratios were calculated by forming the ratio of the mismatch or perfect match signal intensity to the perfect match signal intensity within one SNP probe set and calculating the mean between n times three probe sets, herein referred to as “relative intensity” (RI) of one oligo probe [RI_{MM}=I_{MM}/I_{PM} and RI_{PM}=I_{PM}/I_{PM}] (n=number of arrays). Accordingly, all the perfect match relative intensities RI_{PM} corresponded to a value of 1, the mismatch relative intensities RI_{MM} were below 1.

Results

In order to establish a system, which is as reliable as and easily compatible with the already designed TEM microarray (Grimm, V., S. Ezaki, M. Susa, C. Knabbe, R. D. Schmid, and T. T. Bachmann. 2004. Use of DNA microarrays for rapid genotyping of TEM beta-lactamases that confer resistance. J. Clin. Microbiol. 42:3766-3774), an allele specific hybridization format was also chosen for the SHV microarray. In case of the SHV probe design however the traditional allele specific probe design proved to be not sufficient, so that an alternative strategy had to be devised.

### Example 4

Modified Probe Design

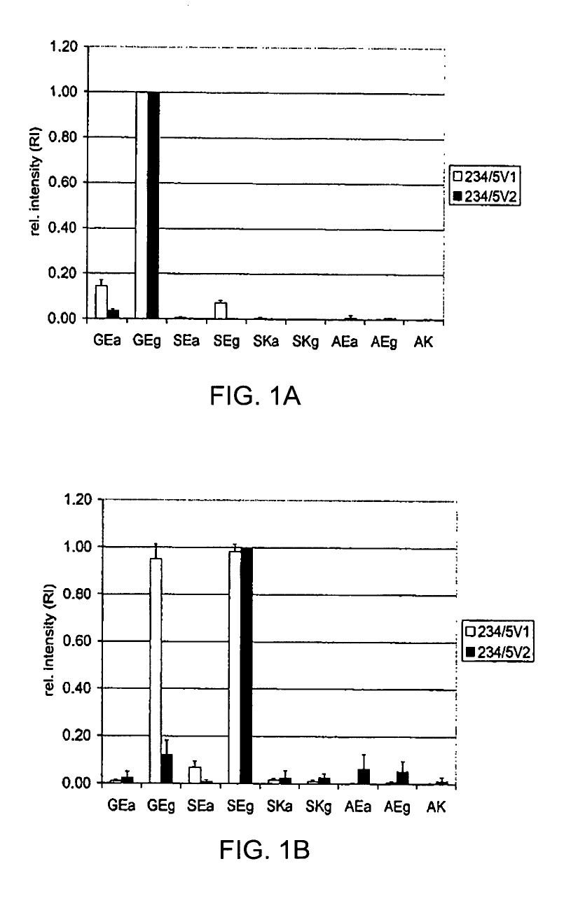

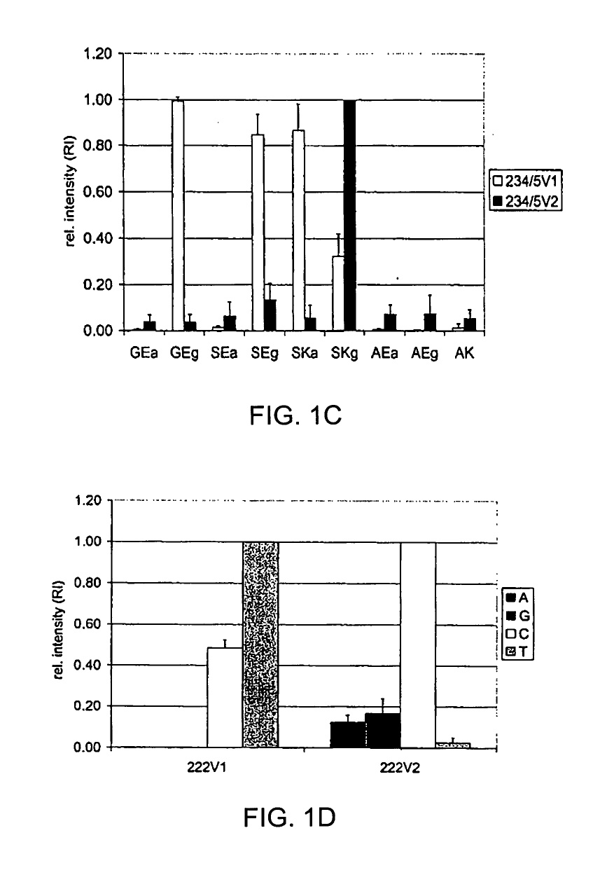

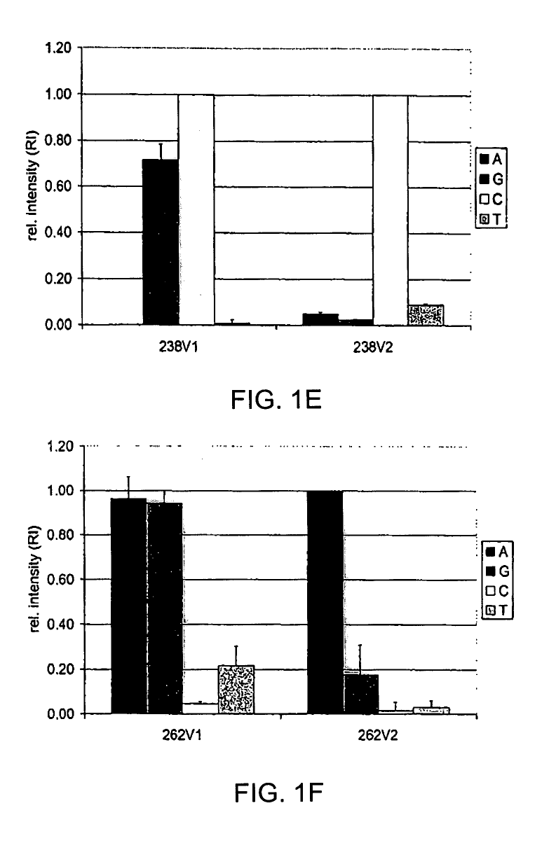

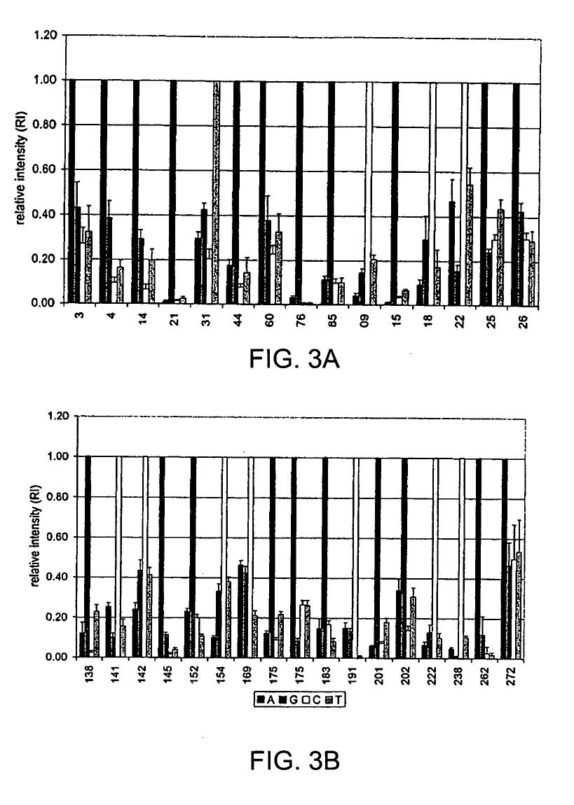

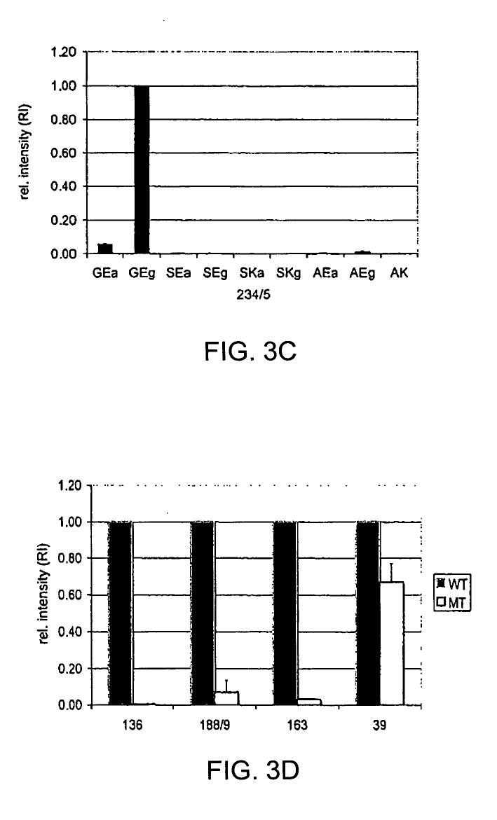

Allele specific hybridization probe sets were designed covering 37 of the 39 mutation positions. Amino acid positions 50 and 171 were excluded from the probe design, because of unfavorable probe secondary structure. Both positions are not necessary for unambiguous identification of the SHV beta-lactamase, which carry a mutation at the concerned positions: variants (SHV-9, -10, -51), since position 141 uniquely identifies SHV-51 and position 136 also targets SHV-9, -10. For amino acid positions 39, 222, 234/5, 238, and 262 the calculated dimer dG results for the first probe versions, which were designed (see dG values Table 1) were unfavorable. Usually dimers with a calculated dG of more than −6 kcal/mol are excluded from the probe design. However since this array should include the relevant mutation positions for unequivocal identification of all naturally occurring SHV variants, these probe positions could not be excluded. The hybridization results confirmed the theoretical data. The relative intensities of probe versions 1 are displayed in FIG. 1. For position 234/5 the identification of the correct bla_{SHV-1} codons (GEg) was possible, but for bla_{SHV-2} (SEg) and bla_{SHV-4} (SKg) the relative intensity of the GEg probe was as high or even higher as the real PM (perfect match). This corresponds to a dimer dG of −6.2 kcal/mol for SEg or SKg in contrast to a dG of only −2.9 kcal/mol for the GEg probe. Thus the better accessibility of the GEg probe compensates the effect of the steric hindrance by the mismatched central base. Due to the same reasons, in case of probe 222 and 262 the wrong position was identified as a perfect match (perfect match of probe 222 is C, but T is identified due to higher signal intensity, for 262 A is the perfect match instead of G). For position 238 a high relative intensity of the mismatch position G (RI_{MM}=0.72) could be observed. The dimer forming bases were in all cases in close proximity to the mutation position and shifting of the mutation position within the probe sequence only led to even worse secondary structures or less efficient discriminatory power. At position 262, the mutation is shifted from the center to the 5′ end of probe version 1 since with a central SNP a highly stable hairpin structure can be formed in addition to the already existing dimer (data not shown). Therefore a new probe design concept had to be developed. It was assumed, that through introduction of an additional mismatch the secondary structure can be dissolved, while retaining enough hybridization capability to allow the MM/PM discrimination. In table 1 the bases, which were exchanged in probe version 2 in comparison to version 1 are underlined. Furthermore the probe positions and length were adjusted for optimal accessibility of the new probes. In case of probe 222V2, 234/5V2, 238V2 and 262V2 the relative intensities allowed a highly specific identification of the correct perfect match position in contrast to probe version 1 (see FIG. 1). The perfect match net signal intensities were diminished due to the additional mismatched base, but remained at a sufficient level of at least 10× above background net signal intensity. These probes were included in the SHV array layout. In case of probe 39 it was not possible to design a new probe according to the new probe concept, because at least two mismatched bases are necessary to release the very unfavorable dimer structure of the probe 39C and as expected no signals could be obtained with this probe 39V2 (data not shown). R is of probe 39V1 showed a higher MM fluorescence signal for probe 39T in comparison to the correct SHV-1 perfect match position 39C (data not shown). However the 39C SHV-1 PM can be identified correctly, when compared to the RI of probe 39A, which is the PM for the mutant SHVs. For a more comprehensive PM identification in this case the 4 probe concept per SNP was skipped and only probe 39C (alias 39WT) in comparison to 39A (alias 39MT) was considered (see FIG. 3c). Thus a specific identification of the mutant SHV or SHV-1 was possible (see results of variant identification).

Proof of Concept

As a proof of concept it was shown, that all perfect match positions could be identified for a hybridization with 200 ng SHV-1 target DNA, which was derived from a laboratory culture of E. coli DH5a bearing a pCCR9 plasmid. The target DNA with a NT/F ratio of 55 was hybridized for one hour under medium agitation in a hybridization station. It was the aim to assess the specificity and the reproducibility of the PM detection of the SHV array under standard conditions. All perfect match positions for SHV-1 were correctly identified. The absolute net signal intensities ranged from 2400 to 46000 for the probes with PMs (data not shown). The RIs were calculated to investigate the discriminatory power of the system. The mean RIs and standard deviations for all mutation positions are shown in FIG. 3. 97% of the RI_{MM} remained less than 0.5. The highest RI_{MM} was detected for probe 39MT with 0.67. The standard deviations for the mean RI_{MM} ranged from 0.00 to 0.17; more than 92% of the RI_{MM} values remained below 10% of the RI_{PM} value.

Identification of SHV Variants

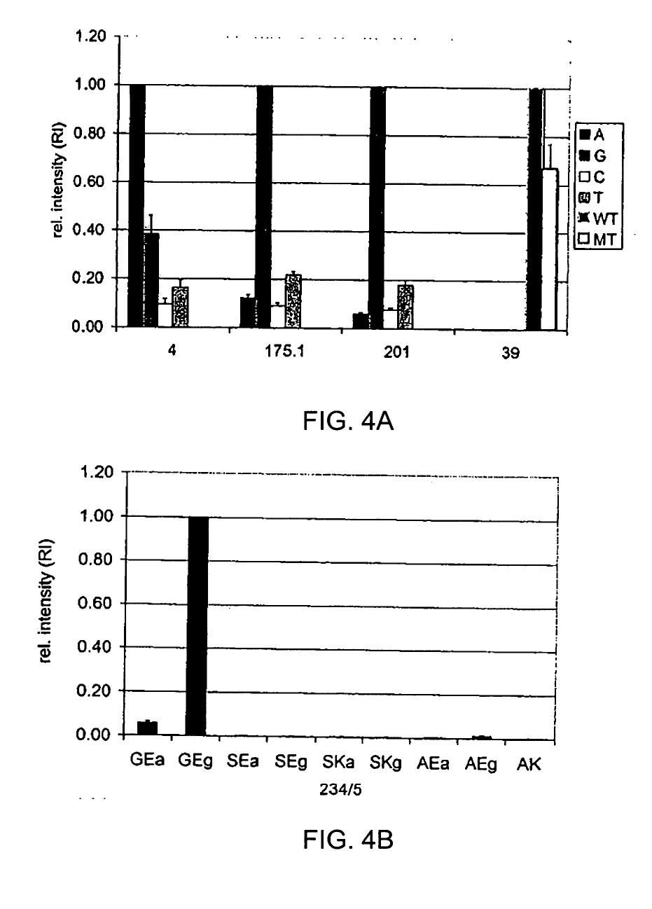

The performance of the array was tested with a set of genetically and phenotypically well characterized SHV gene variants (Randegger, C. C., A. Keller, M. Irla, A. Wada, and H. Hachler. 2000. Contribution of natural amino acid substitutions in SHV extended-spectrum beta-lactamases to resistance against various beta-lactams. Antimicrobial Agents and Chemotherapy 44:2759-2763). Specifically the investigated SHV variants were SHV-1, -2, -3, -4, -5, -7 and -8. The results of the identification of bla_{SHV-1} (n=3), bla_{SHV-2} (n=3), bla_{SHV-3} (n=3), bla_{SHV-4} (n=3), bla_{SHV-5} (n=3), bla_{SHV-7} (n=3) and bla_{SHV-8} (n=3) are given as RI values in FIG. 4. Only the mutation positions, which varied in comparison to SHV-1 are displayed. All correct PMs were identified without ambiguity. The values for 97% of the MM positions remained below an RI_{MM} limit of 0.5. Only 3% of the RI_{MM} values ranged from 0.4 to 0.7. Two times the RI_{MM} limit of 0.7 was violated by probe 39MT with RI_{MM} values up to 0.78 for the identification of SHV-2 and -4. In contrast to that the identification of SHV-7 showed, that the detection of a mutated position 39 in the target is highly specific, since then the RI_{MM} value of the 39WT probe is then less than 0.1 (see FIG. 4f). The standard deviations of the mean RI_{MM} values varied from 0.00 to 0.33, but 92% of the values remained below 10% of RI_{PM} (data not shown). The perfect match position of probe 262 could only be identified for SHV-1 (data not shown), since here the probe 262 C targets the SHV-1 codon (ACC) and probe 262T targets the codon for SHV-39 (TCC), excluding other SHV-sequences, which carry a silent mutation (ACG for SHV-2, -3, -4, -5, -7, -8). Here the probe net signal intensities are not above background level and not relevant for identification, so that they are excluded from the RI calculation of those SHV variants. SHV-3 and SHV-4 carry a mutation at position 201, due to that the net signal intensities of the neighboring probe 202 are not above background level and are also excluded from the RI calculation. However the detected positions allow an unequivocal identification of each tested SHV variant.

### Example 5

Detection of bla_{SHV} Genes in Clinical Isolates

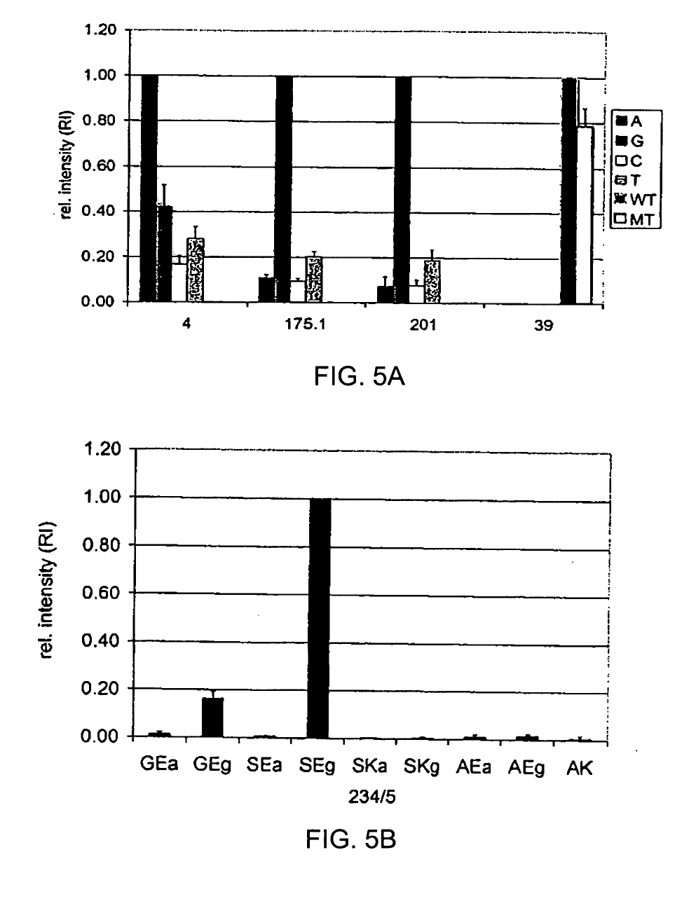

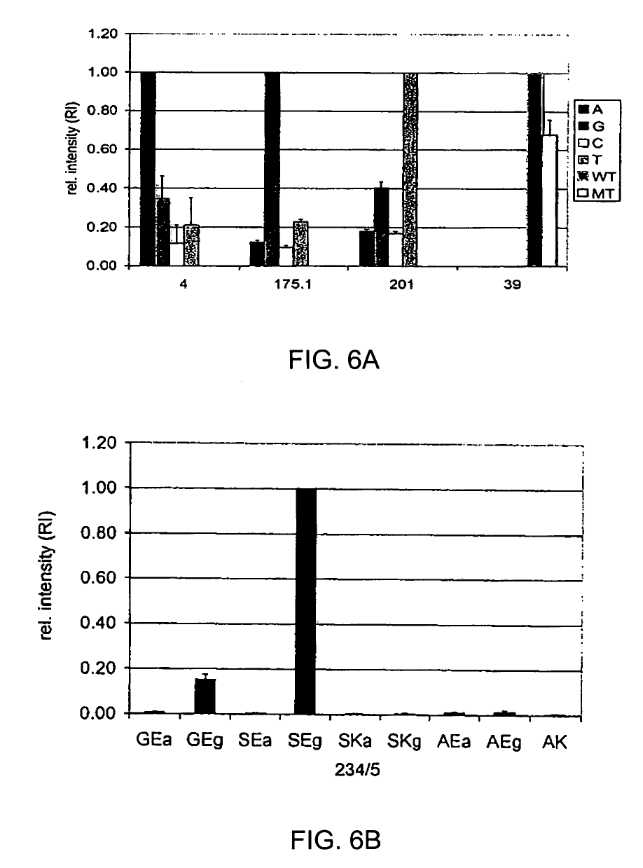

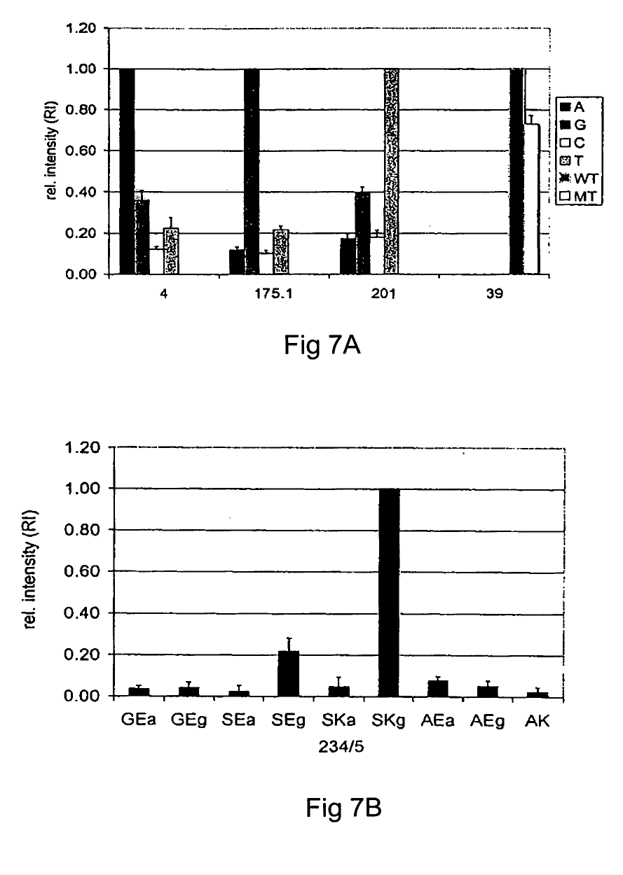

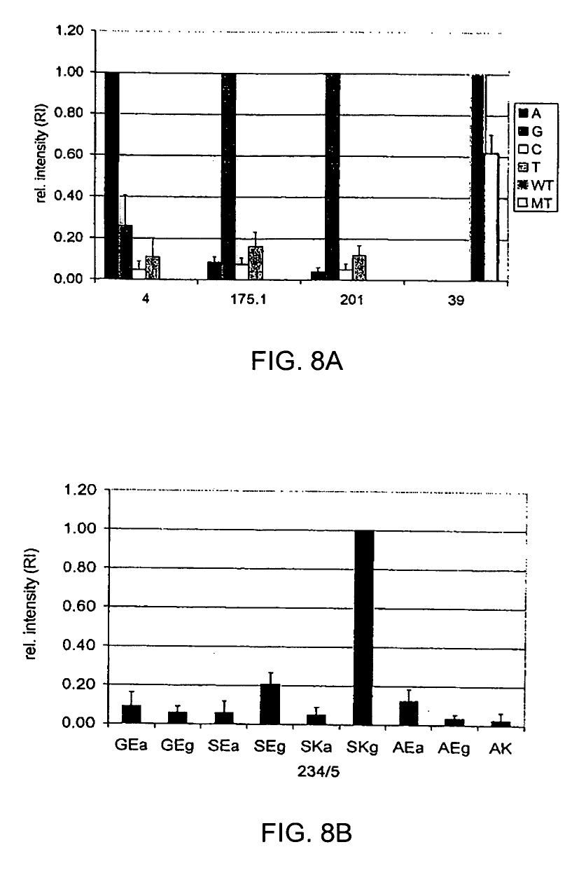

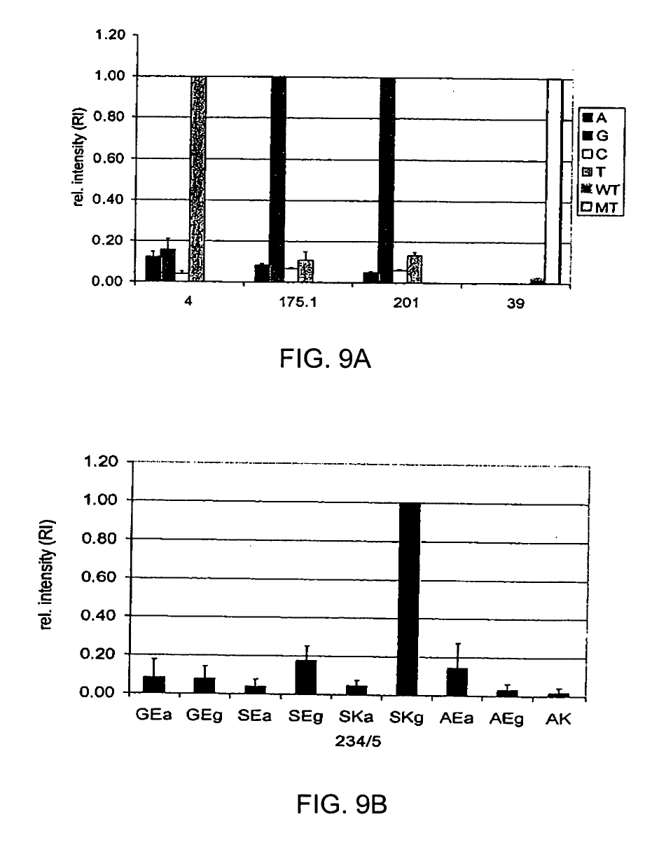

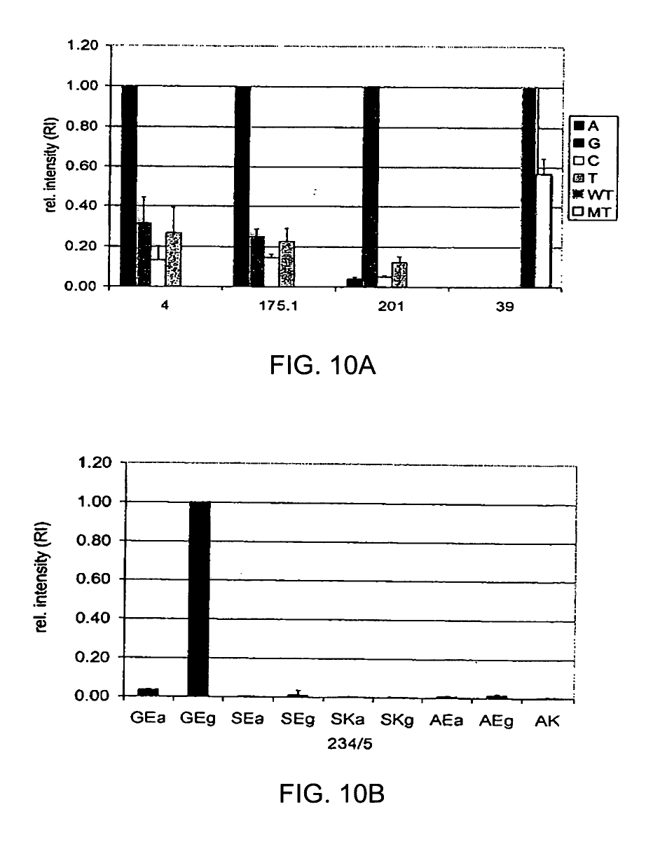

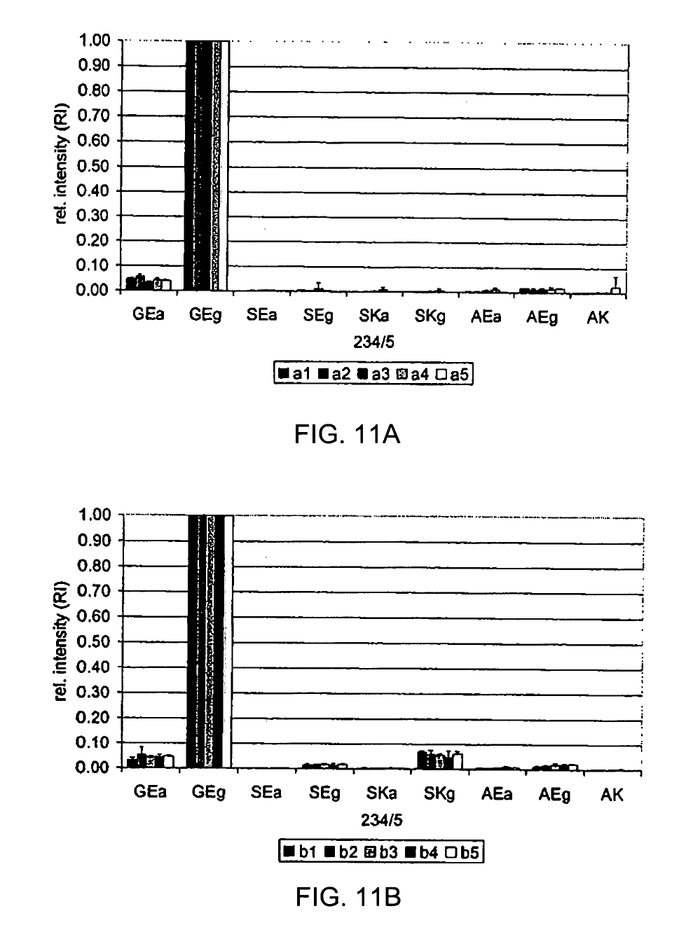

For the testing of clinical isolates it must be considered that Klebsiella pneumoniae isolates are likely to bear a chromosomally encoded SHV-1 (Babini, G. S. and D. M. Livermore. 2000. Are SHV beta-lactamases universal in Klebsiella pneumoniae? Antimicrobial Agents and Chemotherapy 44:2230). The target DNA used in this study originated from alkaline lysed plasmid preparations, but it is possible, that these preparations still contain also bla_{SHV-1} DNA in addition to the ESBL variant gene. To assess the capability of the system to detect a plasmid variant in presence of SHV-1 a set of five different isolates of K. pneumoniae was tested. It was determined that these isolates contain SHV-5 in addition to SHV-1, by the hybridization pattern of position 234/5 (for R is see FIG. 5b). In addition to the high signal of position 234/5 GEg a signal on SKg could be detected with a net signal intensity ranging from 1100-2000 for the five isolates, which was at least 9× above background net intensity level (data not shown). In case of hybridisation of target DNA containing pure 234/5 GEg (RIs shown in FIG. 5a for five independent hybridisations of target DNAs from different amplification reactions) the net signal intensities of SKg ranged from 9-36 and thus no signal above background level was detected for probe SKg (data not shown). The relatively high signal intensity of probe GEg in comparison to SKg is probably mainly due to the differing Tm (see table 2). However the high reproducibility of the system and the fact that the detected signals are clearly above background level for the ESBL variant SKg probe allows a definite identification of an ESBL variant in presence of SHV-1.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A method for genotyping SHV beta lactamase by determining the presence or absence of at least one single nucleotide polymorphism, said method comprising the steps of:
(a) providing target DNA of SHV beta lactamase to be analyzed;
(b) amplifying and labeling said target DNA;
(c) fragmenting said amplified and labeled target DNA into smaller fragments;
(d) contacting said fragments with an array of oligonucleotide capture probes, said array of oligonucleotide capture probes having at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe;
(e) hybridizing said fragments of target DNA to said array of oligonucleotide probes;
(f) determining the presence of a hybridization of a target DNA fragment to said at least one first oligonucleotide probe, said hybridization to said first oligonucleotide capture probe being indicative for the presence of SHV beta lactamase without single nucleotide polymorphism at the position of said at least one nucleotide of interest;
(g) determining the presence of a hybridization of a target DNA fragment to said at least one second oligonucleotide probe, said hybridization to said second oligonucleotide capture probe being indicative for the presence of a SHV beta lactamase having said at least one single nucleotide polymorphism at the position of said at least one nucleotide of interest; and
(h) determining the presence or absence of a specific genotype of SHV beta lactamase on the basis of the results of steps (f) and (g).

2. The method according to claim 1, wherein steps (a) to (h) are performed using instead of said at least one second oligonucleotide capture probe comprising an oligonucleotide capture sequence differing in at least one nucleotide of interest from said oligonucleotide capture sequence of said first oligonucleotide capture probe at least one third oligonucleotide capture probe comprising an oligonucleotide capture sequence differing in at least one nucleotide of interest from said oligonucleotide capture sequence of said first and second oligonucleotide capture probe.

3. The method according to claim 1, wherein said capture sequence capable of hybridizing specifically to a sequence of bla_{SHV} gene of a SHV beta lactamase is an oligonucleotide sequence completely complementary to a sequence of bla_{SHV} gene of SHV beta lactamase.

4. The method according to claim 1, wherein in said step of fragmenting said amplified and labeled target DNA, a mixture comprising said smaller fragments is obtained, which mixture of fragments is contacted with said array of oligonucleotide probes.

5. The method according to claim 1, wherein said label is selected from the group consisting of a dye and a radioactive label.

6. The method according to claim 1, wherein said fragments of amplified and labeled target DNA have a size of 15 to about 150 bp (base pairs).

7. The method according to claim 1, wherein said labeling is performed simultaneously with said amplifying step by incorporating a labeled nucleotide into said amplified DNA.

8. The method according to claim 1, wherein said SHV beta lactamase is selected from the group consisting of a Klebsiella pneumoniae beta lactamase, a Escherichia coli beta lactamase and a Klebsiella pneumoniae beta lactamase bearing a chromosomally encoded SHV.

9. The method according to claim 1, said method further comprising an identification of the presence of one or more TEM beta lactamase genotypes.

10. A method of determining antibiotic resistance conferred by a SHV-type Extended Spectrum Beta Lactamase, said method comprising the steps of genotyping SHV beta lactamase according to a method for genotyping SHV beta lactamase by determining the presence or absence of at least one single nucleotide polymorphism, said method comprising the steps of:
(a) providing target DNA of SHV beta lactamase to be analyzed;
(b) amplifying and labeling said target DNA;
(c) fragmenting said amplified and labeled target DNA into smaller fragments;
(d) contacting said fragments with an array of oligonucleotide capture probes, said array of oligonucleotide capture probes having at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe;
(e) hybridizing said fragments of target DNA to said array of oligonucleotide probes;
(f) determining the presence of a hybridization of a target DNA fragment to said at least one first oligonucleotide probe, said hybridization to said first oligonucleotide capture probe being indicative for the presence of SHV beta lactamase without single nucleotide polymorphism at the position of said at least one nucleotide of interest;
(g) determining the presence of a hybridization of a target DNA fragment to said at least one second oligonucleotide probe, said hybridization to said second oligonucleotide capture probe being indicative for the presence of a SHV beta lactamase having said at least one single nucleotide polymorphism at the position of said at least one nucleotide of interest;
(h) determining the presence or absence of a specific genotype of SHV beta lactamase on the basis of the results of steps (f) and (g); and
(i) determining the presence or absence of an antibiotic resistance depending on the presence or absence of a SHV beta lactamase genotype associated with an antibiotic resistance.

11. A method for designing an oligonucleotide capture probe having an oligonucleotide capture sequence, said method comprising:
determining an oligonucleotide capture probe having an oligonucleotide capture sequence completely complementary to a sequence of bla_{SHV} gene of SHV beta lactamase but having an insufficient capability of specifically hybridizing to said sequence of bla_{SHV}, and
adapting said oligonucleotide capture probe by replacing at least one nucleotide by another naturally occurring nucleotide or a nucleotide equivalent in said oligonucleotide capture sequence.

12. An isolated oligonucleotide having a sequence as identified in SEQ. ID. NO. 1 to 98, or a molecule comprising a sequence as identified in SEQ. ID. NO. 1 to 98, and at least one spacer moiety attached to at least one end of said oligonucleotide.

13. Isolated oligonucleotide or molecule according to claim 12, said isolated oligonucleotide or said molecule being equipped with a spacer consisting of thymidine moieties.

14. An oligonucleotide array comprising,
at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe; and
said at least one first oligonucleotide capture probe being present on said at least one support in a first localized area, and said at least one second oligonucleotide capture probe being present on said at least one support in a second localized area.

15. The oligonucleotide array according to claim 14, wherein said at least one second oligonucleotide capture probe comprises a sequence selected from the group consisting of sequences having SEQ ID NO: 1 to 94.

16. The oligonucleotide array according to claim 14, wherein the support is a slide.

17. The oligonucleotide array according to claim 14, said oligonucleotide array includes at least one of the following controls selected from the group consisting of a spotting control, a positive hybridization control, a negative hybridization control and a process control, said spotting control, said positive hybridization control and said negative hybridization control consisting of sequences unrelated to bacterial species, said process control corresponding to a conserved sequence within the bla_{SHV} gene family.

18. A kit comprising the oligonucleotide array according to an oligonucleotide array comprising,
at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe; and
said at least one first oligonucleotide capture probe being present on said at least one support in a first localized area, and said at least one second oligonucleotide capture probe being present on said at least one support in a second localized area and at least one of reaction reagent, enzyme(s), and supporting material, and instruction leaflet for carrying out an assay.

19. An amplification primer for amplifying the bla_{SHV} gene, said amplification primer having a nucleotide sequence as identified in SEQ.ID. NO: 99 or 100.

20. Use of an oligonucleotide or use of a molecule according to claim 12 as a capture probe.

21. The method according to claim 1, wherein said label is a fluorescence detection label.

22. A method for designing an oligonucleotide capture probe having an oligonucleotide capture sequence according to claim 11, comprising:
adapting the length of said oligonucleotide capture sequence by deleting one or more nucleotides present in terminal position in the oligonucleotide capture sequence or by adding one or more nucleotides at terminal position.

23. The oligonucleotide array according to claim 14, wherein at least ten different further oligonucleotide probes comprising each a different sequence selected from the group consisting of sequences having SEQ ID NO: 1 to 94 are present.

24. The oligonucleotide array according to claim 14, wherein the support is an epoxy coated glass slide.

25. A kit according to claim 18 for performing a method for genotyping SHV beta lactamase by determining the presence or absence of at least one single nucleotide polymorphism, said method comprising the steps of:
(a) providing target DNA of SHV beta lactamase to be analyzed;
(b) amplifying and labeling said target DNA;
(c) fragmenting said amplified and labeled target DNA into smaller fragments;
(d) contacting said fragments with an array of oligonucleotide capture probes, said array of oligonucleotide capture probes having at least one first oligonucleotide capture probe comprising an oligonucleotide capture sequence capable of hybridizing specifically to a sequence of bla_{SHV} gene of SHV beta lactamase and at least one second oligonucleotide capture probe comprising an oligonucleotide capture sequence differing in at least one nucleotide of interest from said oligonucleotide capture sequence of said first oligonucleotide capture probe;
(e) hybridizing said fragments of target DNA to said array of oligonucleotide probes;
(f) determining the presence of a hybridization of a target DNA fragment to said at least one first oligonucleotide probe, said hybridization to said first oligonucleotide capture probe being indicative for the presence of SHV beta lactamase without single nucleotide polymorphism at the position of said at least one nucleotide of interest;
(g) determining the presence of a hybridization of a target DNA fragment to said at least one second oligonucleotide probe, said hybridization to said second oligonucleotide capture probe being indicative for the presence of a SHV beta lactamase having said at least one single nucleotide polymorphism at the position of said at least one nucleotide of interest; and
(h) determining the presence or absence of a specific genotype of SHV beta lactamase on the basis of the results of steps (f) and (g).


SEQUENCE LISTING (100 sequences)

SEQ ID NO: 1 - DNA, 16 bp, Artificial
  Chemically synthesized
tgtcgggcagcgtagg

SEQ ID NO: 2 - DNA, 16 bp, Artificial
  Chemically synthesized
tgtcgggcggcgtagg

SEQ ID NO: 3 - DNA, 16 bp, Artificial
  Chemically synthesized
tgtcgggccgcgtagg

SEQ ID NO: 4 - DNA, 16 bp, Artificial
  Chemically synthesized
tgtcgggctgcgtagg

SEQ ID NO: 5 - DNA, 15 bp, Artificial
  Chemically synthesized
cgtgctgacggcggg

SEQ ID NO: 6 - DNA, 15 bp, Artificial
  Chemically synthesized
cgtgctggcggcggg

SEQ ID NO: 7 - DNA, 15 bp, Artificial
  Chemically synthesized
cgtgctgccggcggg

SEQ ID NO: 8 - DNA, 15 bp, Artificial
  Chemically synthesized
cgtgctgtcggcggg

SEQ ID NO: 9 - DNA, 14 bp, Artificial
  Chemically synthesized
ccgtgctgactgcg

SEQ ID NO: 10 - DNA, 14 bp, Artificial
  Chemically synthesized
ccgtgctggctgcg

SEQ ID NO: 11 - DNA, 14 bp, Artificial
  Chemically synthesized
ccgtgctgcctgcg

SEQ ID NO: 12 - DNA, 14 bp, Artificial
  Chemically synthesized
ccgtgctgtctgcg

SEQ ID NO: 13 - DNA, 16 bp, Artificial
  Chemically synthesized
ggagctggcgaacggg

SEQ ID NO: 14 - DNA, 16 bp, Artificial
  Chemically synthesized
ggagctggcgagcggg

SEQ ID NO: 15 - DNA, 16 bp, Artificial
  Chemically synthesized
ggagctagcgaacggg

SEQ ID NO: 16 - DNA, 16 bp, Artificial
  Chemically synthesized
ggagctagcgagcggg

SEQ ID NO: 17 - DNA, 16 bp, Artificial
  Chemically synthesized
ggagctagcaaacggg

SEQ ID NO: 18 - DNA, 16 bp, Artificial
  Chemically synthesized
ggagctagcaagcggg

SEQ ID NO: 19 - DNA, 16 bp, Artificial
  Chemically synthesized
ggagctgccgaacggg

SEQ ID NO: 20 - DNA, 16 bp, Artificial
  Chemically synthesized
ggagctgccgagcggg

SEQ ID NO: 21 - DNA, 16 bp, Artificial
  Chemically synthesized
ggagctgccaaacggg

SEQ ID NO: 22 - DNA, 15 bp, Artificial
  Chemically synthesized
ggagctggtgaacgg

SEQ ID NO: 23 - DNA, 15 bp, Artificial
  Chemically synthesized
ggagctggtgagcgg

SEQ ID NO: 24 - DNA, 15 bp, Artificial
  Chemically synthesized
ggagctagtgaacgg

SEQ ID NO: 25 - DNA, 15 bp, Artificial
  Chemically synthesized
ggagctagtgagcgg

SEQ ID NO: 26 - DNA, 15 bp, Artificial
  Chemically synthesized
ggagctagtaaacgg

SEQ ID NO: 27 - DNA, 15 bp, Artificial
  Chemically synthesized
ggagctagtaagcgg

SEQ ID NO: 28 - DNA, 15 bp, Artificial
  Chemically synthesized
ggagctgctgaacgg

SEQ ID NO: 29 - DNA, 15 bp, Artificial
  Chemically synthesized
ggagctgctgagcgg

SEQ ID NO: 30 - DNA, 15 bp, Artificial
  Chemically synthesized
ggagctgctaaacgg

SEQ ID NO: 31 - DNA, 16 bp, Artificial
  Chemically synthesized
ggggtgagcgcgggat

SEQ ID NO: 32 - DNA, 16 bp, Artificial
  Chemically synthesized
ggggtgggcgcgggat

SEQ ID NO: 33 - DNA, 16 bp, Artificial
  Chemically synthesized
ggggtgtgcgcgggat

SEQ ID NO: 34 - DNA, 16 bp, Artificial
  Chemically synthesized
ggggtgcgcgcgggat

SEQ ID NO: 35 - DNA, 15 bp, Artificial
  Chemically synthesized
cggggtgagtgcggg

SEQ ID NO: 36 - DNA, 15 bp, Artificial
  Chemically synthesized
cggggtgggtgcggg

SEQ ID NO: 37 - DNA, 15 bp, Artificial
  Chemically synthesized
cggggtgcgtgcggg

SEQ ID NO: 38 - DNA, 15 bp, Artificial
  Chemically synthesized
cggggtgtgtgcggg

SEQ ID NO: 39 - DNA, 15 bp, Artificial
  Chemically synthesized
gggataccccggcga

SEQ ID NO: 40 - DNA, 15 bp, Artificial
  Chemically synthesized
gggatgccccggcga

SEQ ID NO: 41 - DNA, 15 bp, Artificial
  Chemically synthesized
gggatcccccggcga

SEQ ID NO: 42 - DNA, 15 bp, Artificial
  Chemically synthesized
gggattccccggcga

SEQ ID NO: 43 - DNA, 15 bp, Artificial
  Chemically synthesized
ctgcgtgataccccg

SEQ ID NO: 44 - DNA, 15 bp, Artificial
  Chemically synthesized
ctgcgtgatgccccg

SEQ ID NO: 45 - DNA, 15 bp, Artificial
  Chemically synthesized
ctgcgtgatcccccg

SEQ ID NO: 46 - DNA, 15 bp, Artificial
  Chemically synthesized
ctgcgtgattccccg

SEQ ID NO: 47 - DNA, 19 bp, Artificial
  Chemically synthesized
atgcgttntattcgcctgt

SEQ ID NO: 48 - DNA, 19 bp, Artificial
  Chemically synthesized
atgcgttatnttcgcctgt

SEQ ID NO: 49 - DNA, 18 bp, Artificial
  Chemically synthesized
ctgttagccnccctgccg

SEQ ID NO: 50 - DNA, 16 bp, Artificial
  Chemically synthesized
cggtacacnccagccc

SEQ ID NO: 51 - DNA, 24 bp, Artificial
  Chemically synthesized
agcaaattaaacnaagcgaaagcc

SEQ ID NO: 52 - DNA, 16 bp, Artificial
  Chemically synthesized
tgtcgggcngcgtagg

SEQ ID NO: 53 - DNA, 25 bp, Artificial
  Chemically synthesized
gtaggcatgatanaaatggatctgg

SEQ ID NO: 54 - DNA, 18 bp, Artificial
  Chemically synthesized
cgccgatgnacgctttcc

SEQ ID NO: 55 - DNA, 15 bp, Artificial
  Chemically synthesized
cggcgcantgctggc

SEQ ID NO: 56 - DNA, 17 bp, Artificial
  Chemically synthesized
gccggtgacnaacagct

SEQ ID NO: 57 - DNA, 21 bp, Artificial
  Chemically synthesized
tcgaaaaacacnttgccgacg

SEQ ID NO: 58 - DNA, 17 bp, Artificial
  Chemically synthesized
gcatgacgntcggcgaa

SEQ ID NO: 59 - DNA, 16 bp, Artificial
  Chemically synthesized
gtcggcgaantctgcg

SEQ ID NO: 60 - DNA, 16 bp, Artificial
  Chemically synthesized
cgccgccgncattacc

SEQ ID NO: 61 - DNA, 25 bp, Artificial
  Chemically synthesized
ccgccattaccnttagcgataacag

SEQ ID NO: 62 - DNA, 23 bp, Artificial
  Chemically synthesized
ccattaccatgngcgataacagc

SEQ ID NO: 63 - DNA, 20 bp, Artificial
  Chemically synthesized
ctgctactggccaccgtcgg

SEQ ID NO: 64 - DNA, 20 bp, Artificial
  Chemically synthesized
ctgctactccggaccgtcgg

SEQ ID NO: 65 - DNA, 15 bp, Artificial
  Chemically synthesized
ggccaccntcggcgg

SEQ ID NO: 66 - DNA, 15 bp, Artificial
  Chemically synthesized
gcggccncgcaggat

SEQ ID NO: 67 - DNA, 16 bp, Artificial
  Chemically synthesized
cggccccgnaggattg

SEQ ID NO: 68 - DNA, 16 bp, Artificial
  Chemically synthesized
aggattgnctgccttt

SEQ ID NO: 69 - DNA, 17 bp, Artificial
  Chemically synthesized
ccagatcgncgacaacg

SEQ ID NO: 70 - DNA, 17 bp, Artificial
  Chemically synthesized
cggcgacaangtcaccc

SEQ ID NO: 71 - DNA, 20 bp, Artificial
  Chemically synthesized
ctgggaaacggaactgaatg

SEQ ID NO: 72 - DNA, 20 bp, Artificial
  Chemically synthesized
ctgggaaactgaccgctggg

SEQ ID NO: 73 - DNA, 16 bp, Artificial
  Chemically synthesized
atgaggcgnttcccgg

SEQ ID NO: 74 - DNA, 15 bp, Artificial
  Chemically synthesized
cgcccgcnacaccac

SEQ ID NO: 75 - DNA, 15 bp, Artificial
  Chemically synthesized
gcccgcgncaccact

SEQ ID NO: 76 - DNA, 16 bp, Artificial
  Chemically synthesized
ccagcatgnccgcgac

SEQ ID NO: 77 - DNA, 17 bp, Artificial
  Chemically synthesized
gcgcaagctgctgacca

SEQ ID NO: 78 - DNA, 17 bp, Artificial
  Chemically synthesized
gcgcaacgtgctgacca

SEQ ID NO: 79 - DNA, 17 bp, Artificial
  Chemically synthesized
gctgctgancagccagc

SEQ ID NO: 80 - DNA, 17 bp, Artificial
  Chemically synthesized
gttcgcaacngcagctg

SEQ ID NO: 81 - DNA, 16 bp, Artificial
  Chemically synthesized
gcaacggcanctgctg

SEQ ID NO: 82 - DNA, 14 bp, Artificial
  Chemically synthesized
ccgtgctgnctgcg

SEQ ID NO: 83 - DNA, 15 bp, Artificial
  Chemically synthesized
ggagctggtgagcgg

SEQ ID NO: 84 - DNA, 15 bp, Artificial
  Chemically synthesized
ggagctggtgaacgg

SEQ ID NO: 85 - DNA, 15 bp, Artificial
  Chemically synthesized
ggagctagtgaacgg

SEQ ID NO: 86 - DNA, 15 bp, Artificial
  Chemically synthesized
ggagctagtgagcgg

SEQ ID NO: 87 - DNA, 15 bp, Artificial
  Chemically synthesized
ggagctagtaaacgg

SEQ ID NO: 88 - DNA, 15 bp, Artificial
  Chemically synthesized
ggagctagtaagcgg

SEQ ID NO: 89 - DNA, 15 bp, Artificial
  Chemically synthesized
ggagctgctgaacgg

SEQ ID NO: 90 - DNA, 15 bp, Artificial
  Chemically synthesized
ggagctgctgagcgg

SEQ ID NO: 91 - DNA, 15 bp, Artificial
  Chemically synthesized
ggagctgctaaacgg

SEQ ID NO: 92 - DNA, 15 bp, Artificial
  Chemically synthesized
cggggtgngtgcggg

SEQ ID NO: 93 - DNA, 15 bp, Artificial
  Chemically synthesized
ctgcgtgatnccccg

SEQ ID NO: 94 - DNA, 19 bp, Artificial
  Chemically synthesized
aaatcagcanatcgccggg

SEQ ID NO: 95 - DNA, 21 bp, Artificial
  Chemically synthesized
tttaaagtagtgctctgcggc

SEQ ID NO: 96 - DNA, 18 bp, Artificial
  Chemically synthesized
tctagacagccactcata

SEQ ID NO: 97 - DNA, 19 bp, Artificial
  Chemically synthesized
gattggacgagtcaggagc

SEQ ID NO: 98 - DNA, 18 bp, Artificial
  Chemically synthesized
tctagacagccactcata

SEQ ID NO: 99 - DNA, 19 bp, Artificial
  Chemically synthesized
gcaaaacgccgggttattc

SEQ ID NO: 100 - DNA, 19 bp, Artificial
  Chemically synthesized
ggttagcgttgccagtgct